(19) United States
(12) Patent Application Publication
(10) Pub. No.: US 2026/0194533 A1
(43) Pub. Date: Jul. 9, 2026

(54) METHODS AND SYSTEMS FOR IDENTIFYING COMPOUNDS FOR FORMING, STABILIZING OR DISRUPTING MOLECULAR COMPLEXES

(71) Applicant: Enveda Therapeutics, Inc., Boulder, CO (US)

(72) Inventors: Viswa COLLURU, Boulder, CO (US); Gayathree KARTHIKKEYAN, Chennai (IN); Deepanjan GHOSH, Serampore (IN); Biswapriya Biswavas MISRA, New Delhi (IN); Sarah MUBEEN, Ann Arbor, MI (US); Joseph ROKICKI, Louisville, KY (US)

(21) Appl. No.: 19/132,778

(22) PCT Filed: Nov. 22, 2023
(86) PCT No.: PCT/US2023/081000

§ 371 Date: May 23, 2025

(30) Foreign Application Priority Data
Nov. 26, 2022 (IN) 202211068104

(51) Int. Cl. G01N 33/68; G01N 30/88

(52) U.S. Cl. CPC G01N 33/6845; G01N 33/6848; G01N 2030/8831; G01N 2500/02; G01N 2570/00

(57) ABSTRACT

Described herein are methods for identifying components of a protein-protein complex and methods for identifying one or more compounds that cause protein complex formation, stabilization, or dissociation. Also described are systems for performing such methods. The methods can include fractionating a first sample containing a first portion of a biological sample and a second sample containing a second portion of the biological sample combined with a drug, compound library, or natural extract. Elution fractions can be analyzed using proteomic or metabolomic methods to identify one or more binding proteins that form a complex with a target protein, or one or more compounds that cause complex formation, stabilization or dissociation.

### CROSS-REFERENCE TO RELATED APPLICATIONS

This application claims the priority benefit of Indian Application No. 202211068104, filed on Nov. 26, 2022, the contents of which is incorporated herein by reference for all purposes.

### TECHNICAL FIELD

Described herein are systems and methods for identifying components of a protein-protein complex. Also described herein are systems and methods for identifying compounds that cause complex formation, stabilization, or dissociation.

### BACKGROUND

Protein-protein interactions (PPIs) are central to a variety of biological processes, and their dysfunction is implicated in the pathogenesis of a range of human diseases and disorders. The contact interface between two proteins is the structural foundation of their interaction. Similar or overlapping protein interfaces can be promiscuous and be employed many times in hub proteins. PPIs may be transient or permanent, identical or heterogeneous, and specific or nonspecific, and can be regulated through signaling (biochemical) cascades. Therefore, the ability to modulate disease-relevant protein-protein interactions (PPIs) using small-molecule inhibitors is an important diagnostic and therapeutic strategy.

Existing techniques used to interrogate regulators of PPIs rely on low-throughput approaches such as “bait and prey” assays. Therefore, there is growing interest developing high throughput, multiplexed approaches that simultaneously interrogate multiple modulators of protein complexes in an unbiased manner.

### SUMMARY OF THE INVENTION

Described herein are systems and methods for identifying components of a protein-protein complex. Also described herein are systems and methods for identifying compounds that cause complex formation, stabilization, or dissociation.

A method for identifying components of a protein-protein complex can include: fractioning a first sample comprising a first portion of a biological sample comprising proteins, using size-exclusion chromatography to generate a first plurality of fractions; fractioning a second sample comprising (i) second portion of the biological sample and (ii) a compound library, a drug, or a natural extract, using the size-exclusion chromatography to generate a second plurality of fractions; analyzing the first plurality of fractions and the second plurality of fractions to identify proteins in the first plurality of fractions and the second plurality of fractions; identifying, for a target protein, a fractionation shift between the first plurality of fractions and the second plurality of fractions, wherein the fraction shift indicates complex formation or stabilization, or complex dissociation, caused by the drug or one or more compounds in the compound library or the natural extract; and identifying one or more binding proteins that form a complex with the target protein. Co-elution of the one or more binding proteins with the target protein in the second plurality of fractions but not the first plurality of fractions indicates that the drug or a compound in the compound library or the natural extract causes the formation or stabilization of a complex comprising the target protein and at least one of the one or more binding proteins. Co-elution of the one or more binding proteins with the target protein in the first plurality of fractions but not the second plurality of fractions indicates that the drug or a compound in the compound library or the natural extract causes the dissociation of a complex comprising the target protein and at least one of the one or more binding proteins. Co-elution may be determined, for example, based on a peak elution fraction for the target protein and the one or more binding proteins. Co-elution of the one or more binding proteins with the target protein may be based, for example, on a peak elution fraction of the one or more binding proteins and a peak elution fraction of the target protein. The method may further include selecting one or more of the one or more binding proteins as a member of the complex based on molecular weights of the one or more putative binding proteins and the target protein and a fraction number for a fraction comprising the target protein and the one or more putative binding proteins.

A method for identifying one or more compounds that cause protein complex formation, stabilization, or dissociation can include: fractioning a first sample comprising a first portion of a biological sample comprising proteins, using size-exclusion chromatography to generate a first plurality of fractions; fractioning a second sample comprising (i) second portion of the biological sample and (ii) a compound library or a natural extract, using the size-exclusion chromatography to generate a second plurality of fractions; analyzing the first plurality of fractions and the second plurality of fractions to identify proteins in the first plurality of fractions and the second plurality of fractions; identifying, for a target protein, a fractionation shift between the first plurality of fractions and the second plurality of fractions, wherein the fraction shift indicates complex formation or stabilization, or complex dissociation, caused by one or more compounds in the compound library or the natural extract; and identifying the one or more compounds that cause the fraction shift, comprising analyzing (1) for a fraction shift indicating complex formation or stabilization caused by the one or more compounds, a fraction from the second plurality of fractions that comprises the target protein to identify one or more compounds in the fraction that co-elutes with the target protein, or (2) for a fraction shift indicating complex dissociation caused by the one or more compounds, (i) a fraction from the second plurality of fractions that comprises the target protein to identify one or more compounds in the fraction that co-elutes with the target protein, or (ii) a fraction from the second plurality of fractions that comprises a binding protein that forms a complex with the target protein in the absence of the one or more compounds to identify the one or more compounds in the fraction that co-elutes with the binding protein.

Identifying the one or more compounds that cause the fraction shift may include, for example, obtaining a metabolomics profile for the fraction from the second plurality of fractions; obtaining a metabolomics profile for the compound library or the natural extract; and identifying one or more compounds present in both the fraction and the compound library or the natural extract. In some implementations, identifying the one or more compounds that cause the fraction shift further includes obtaining a metabolomics profile for the first sample; and filtering the metabolomics profile for the fraction from the second plurality of fractions to exclude compounds present in the first sample.

The metabolomics profiles may be obtained using mass spectrometry. For example, in some implementations, the metabolomics profiles are obtained using liquid chromatography and tandem mass spectrometry (LC-MS/MS). In some embodiments, the metabolomics profiles are obtained using in silico nuclear magnetic resonance (NMR).

Identifying the one or more compounds that cause the fraction shift comprises confirming co-elution of the one or more compounds and the target protein or the one or more binding proteins may be based on a peak elution fraction of the one or more compounds and a peak elution fraction of the target protein or the binding protein. In some implementations, the method may include identifying the binding protein that forms the complex with the target protein, wherein co-elution of the binding proteins with the target protein in the first plurality of fractions but not the second plurality of fractions indicates that one or more compounds in the compound library or the natural extract causes the dissociation of a complex comprising the target protein and at the binding protein.

In some implementations of the above methods, analyzing the first plurality of fractions and the second plurality of fractions to identify proteins in the first plurality of fractions and the second plurality of fractions comprises a proteomics analysis. In some implementations of the above methods, analyzing the first plurality of fractions and the second plurality of fractions to identify proteins in the first plurality of fractions and the second plurality of fractions comprises using mass spectrometry. For example, analyzing the first plurality of fractions and the second plurality of fractions to identify proteins in the first plurality of fractions and the second plurality of fractions may include using liquid chromatography with tandem mass spectrometry (LC-MS/MS).

In some implementations of the above methods, the compound library or the natural extract is substantially free of proteins.

In some implementations of the above methods, the biological sample comprises a cell-free biological sample, a tissue extract, a cellular extract, or a sub-cellular extract.

In some implementations of the above methods, the second sample comprises the compound library.

In some implementations of the above methods, the second sample comprises the natural extract.

In some implementations of the above methods, the natural extract is a plant extract.

In some implementations of the above methods, the biological sample comprising proteins is obtained from a cellular lysate.

In some implementations of the above methods, the biological sample comprising proteins is obtained from animal tissue.

In some implementations of the above methods, the biological sample comprising proteins is obtained from mammalian tissue.

In some implementations of the above methods, the biological sample comprising proteins is obtained from brain, liver, lung, or kidney tissue.

In some implementations, the system comprises one or more processors; and a non-transitory computer readable storage medium storing one or more programs that, when executed by the one or more processors, cause the system to: receive a first proteomics profile data for a first plurality of fractions obtained by fractioning, using size-exclusion chromatography, a first sample comprising a portion of a biological sample comprising proteins; receive second proteomics profile data for a second plurality of fractions obtained by fractioning, using the size exclusion chromatography, a second sample comprising (i) second portion of the biological sample and (ii) a compound library, a drug, or a natural extract; identify, based on the first proteomics profile data and the second proteomics data, proteins in the first plurality of fractions and the second plurality of fractions; identify, for a target protein, a fractionation shift between the first plurality of fractions and the second plurality of fractions, wherein the fraction shift indicates complex formation or stabilization, or complex dissociation, caused by the drug or one or more compounds in the compound library or the natural extract; and identify one or more binding proteins that form a complex with the target protein. Co-elution of the one or more binding proteins with the target protein in the second plurality of fractions but not the first plurality of fractions indicates that the drug or a compound in the compound library or the natural extract causes the formation or stabilization of a complex comprising the target protein and at least one of the one or more binding proteins. Co-elution of the one or more binding proteins with the target protein in the first plurality of fractions but not the second plurality of fractions indicates that the drug or a compound in the compound library or the natural extract causes the dissociation of a complex comprising the target protein and at least one of the one or more binding proteins.

In some implementation a system includes one or more processors; and a non-transitory computer readable storage medium storing one or more programs that, when executed by the one or more processors, cause the system to: receive a first proteomics profile data for a first plurality of fractions obtained by fractioning, using size-exclusion chromatography, a first sample comprising a portion of a biological sample comprising proteins; receive second proteomics profile data for a second plurality of fractions obtained by fractioning, using the size exclusion chromatography, a second sample comprising (i) second portion of the biological sample and (ii) a compound library or a natural extract; and identify, based on the first proteomics profile data and the second proteomics data, proteins in the first plurality of fractions and the second plurality of fractions; identify, for a target protein, a fractionation shift between the first plurality of fractions and the second plurality of fractions, wherein the fraction shift indicates complex formation or stabilization, or complex dissociation, caused by one or more compounds in the compound library or the natural extract; receive metabolomics data for the compound library or the natural extract; receive metabolomics data for a fraction, from the second plurality of fractions, comprising the target protein or a binding protein that forms a complex with the target protein in the absence of the one or more compounds; and identify the one or more compounds that cause the fraction shift by analyzing the metabolomics data for the compound library or the natural extract and the metabolomics data for the fraction, from the second plurality of fractions, comprising the target protein or the binding protein that forms a complex with the target protein in the absence of the one or more compounds.

### BRIEF DESCRIPTION OF THE DRAWINGS

FIG. 1A shows an exemplary method for identifying protein-protein interactions, according to some embodiments.

FIG. 1B shows an exemplary method for determining complex formation or complex dissociation, which may be included in the exemplary method shown in FIG. 1A, according to some embodiments.

FIG. 2A shows an exemplary method for identifying a compound that modulates a protein complex, according to some embodiments.

FIG. 2B shows an exemplary method for determining complex formation or complex dissociation, according to some embodiments.

FIG. 3 shows an exemplary schematic of visualized compound-protein interactions, according to some embodiments. Interactions are prioritized based on algorithmic scoring of metabolite-protein interaction confidence.

FIG. 4 shows exemplary methods used to identify components of protein-protein complexes and compounds that cause protein complex formation from pools of known or novel compounds, according to some embodiments.

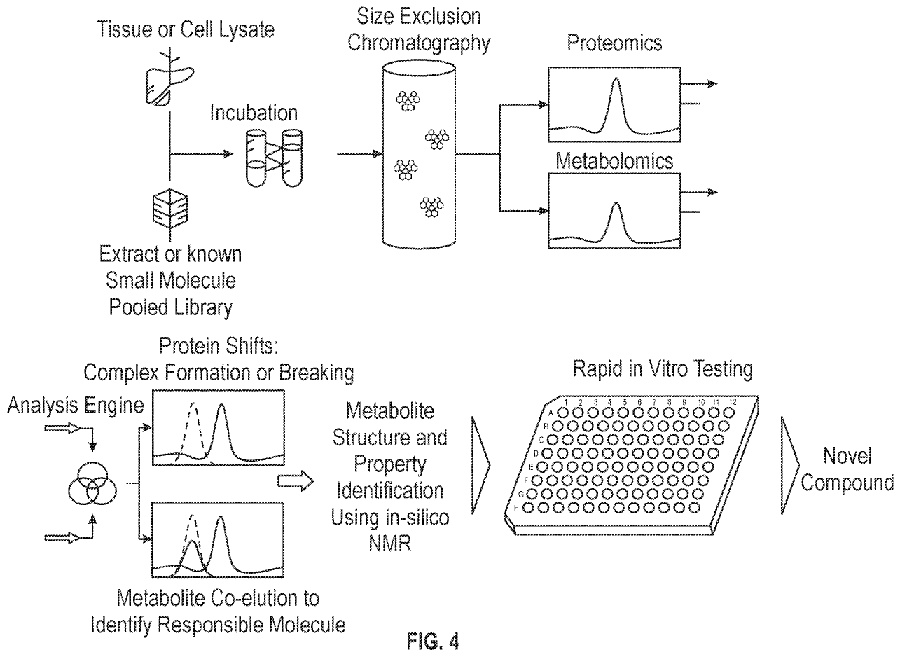

FIG. 5 shows a visualized compound-protein interaction plot of RNF114 with or without natural extract P45, according to an exemplary experiment. Interactions are prioritized based on algorithmic scoring of compound-protein interaction confidence.

FIG. 6 shows a visualized compound-protein interaction plot for RNF114 and binding proteins (gray circle) with or without natural extract P45, according to an exemplary experiment. Interactions are prioritized based on algorithmic scoring of metabolite-protein interaction confidence.

FIG. 7 shows peak analysis plots for RNF114 and a binding protein with or without natural extract P45, according to some embodiments.

FIG. 8 shows an exemplary system that may be used with the methods described herein, according to some embodiments.

FIG. 9 shows an exemplary system used with the method described herein, according to some embodiments.

### DETAILED DESCRIPTION OF THE INVENTION

Described herein are methods and systems for identification of the components of a protein-protein interaction. The proteins in a sample of a biological sample comprising proteins, (e.g., a lysate from a mammalian source such as a tissue, cellular, or sub-cellular lysate, or a cell-free biological sample, such as an extract from saliva, cerebrospinal fluid, plasma, etc.) are divided into fractions based on apparent molecular weight or size. Another portion of the same biological sample is combined with a compound library, natural extract or a drug from a different source (e.g., a plant) and similarly fractioned based on apparent molecular weight or size. The compound library, natural extract or drug combined with the biological sample is preferably protein free or substantially protein free. Proteins that complex with each other migrate together as the complexed apparent size. Fractions are analyzed for the identity of the proteins contained within each fraction.

Each fraction is associated with an elution volume (i.e., a volume of buffer that elutes from a column before the fraction), which can be correlated to the apparent weight or size of a protein (generally assumed by based on a hydrodynamic diameter). A fraction shift is the change in elution volume (or fraction count, which is associated with the elution volume) of a protein under different conditions. As it pertains herein, a fraction shift for a target protein when is the difference in elution volume or fraction count of the target protein in the presence versus absence of the compound library, natural extract, or drug. The fraction shift thus is indicative of protein-protein association (e.g., formation and/or stabilization) or dissociation events caused by the compound library, natural extract, or drug.

Binding proteins may be more confidently identified based on co-migration with the target protein, and the identity of these binding proteins can be identified through proteomics. Described herein are also methods and systems for identifying compounds that cause protein complex formation or stabilization, or dissociation. In some implementations of this method, the proteins in a sample of a biological sample, (e.g., a lysate from a mammalian source such as a tissue, cellular, or sub-cellular extract, or a cell-free biological sample, such as an extract from saliva, cerebrospinal fluid, plasma, etc.) are divided into fractions based on apparent molecular weight or size. Another portion of the same biological sample is combined with a compound library, natural extract or a drug from a different source (e.g., a plant extract) and similarly fractioned based on apparent molecular weight or size. Proteins that complex with each other migrate together as the complexed apparent size. Fractions are divided for further analysis. One part of the fraction is analyzed for the identity of the proteins contained within each fraction. The other part of the fraction is analyzed for the identity of the compounds contained within the fraction.

In some implementations of a method for identifying components of a protein-protein complex, the method includes fractioning a first sample comprising a first portion of a biological sample comprising proteins (e.g., a cell-free biological sample, a tissue extract, a cellular extract, or a sub-cellular extract), using size-exclusion chromatography to generate a first plurality of fractions. A second sample comprising (i) second portion of the biological sample and (ii) a compound library, a drug, or a natural extract, is also fractionated using the size-exclusion chromatography to generate a second plurality of fractions. The first plurality of fractions and the second plurality of fractions are analyzed to identify proteins in the first plurality of fractions and the second plurality of fractions. For a target protein, a fractionation shift between the first plurality of fractions and the second plurality of fractions can then be identified. The fraction shift indicates complex formation or stabilization, or complex dissociation, caused by the drug or one or more compounds in the compound library or the natural extract. One or more binding proteins that form a complex with the target protein can then be identified. For example, co-elution of the one or more binding proteins with the target protein in the second plurality of fractions but not the first plurality of fractions indicates that the drug or a compound in the compound library or the natural extract causes the formation or stabilization of a complex comprising the target protein and at least one of the one or more binding proteins. Co-elution of the one or more binding proteins with the target protein in the first plurality of fractions but not the second plurality of fractions indicates that the drug or a compound in the compound library or the natural extract causes the dissociation of a complex comprising the target protein and at least one of the one or more binding proteins. Co-elution may be determined, for example, based on a peak elution fraction for the target protein and the one or more binding proteins. In some instances, the binding proteins are confirmed as binding proteins. Accordingly, in some implementations, the method further includes selecting one or more of the one or more binding proteins as a member of the complex based on molecular weights of the one or more binding proteins and the target protein and a fraction number for a fraction comprising the target protein and the one or more binding proteins.

A method for identifying one or more compounds that cause protein complex formation or dissociation, can include fractioning a first sample comprising a first portion of a biological sample comprising proteins (e.g., a cell-free biological sample, a tissue extract, a cellular extract, or a sub-cellular extract), using size-exclusion chromatography to generate a first plurality of fractions. A second sample comprising (i) second portion of the biological sample and (ii) a compound library or a natural extract, can also be fractionated using the size-exclusion chromatography to generate a second plurality of fractions. The first plurality of fractions and the second plurality of fractions can be analyzed to identify proteins in the first plurality of fractions and the second plurality of fractions. For a target protein, a fractionation shift between the first plurality of fractions and the second plurality of fractions can be identified. The fraction shift indicates complex formation or stabilization, or complex dissociation, caused by one or more compounds in the compound library or the natural extract. From this the one or more compounds that cause the fraction shift can be identified. For example, for a fraction shift indicating complex formation or stabilization caused by the one or more compounds, a fraction from the second plurality of fractions that comprises the target protein can be analyzed to identify one or more compounds in the fraction that co-elutes with the target protein. For a fraction shift indicating complex dissociation caused by the one or more compounds, (i) a fraction from the second plurality of fractions that comprises the target protein can be analyzed to identify one or more compounds in the fraction that co-elutes with the target protein, or (ii) a fraction from the second plurality of fractions that comprises a binding protein that forms a complex with the target protein in the absence of the one or more compounds can be to identify the one or more compounds in the fraction that co-elutes with the binding protein. Identifying the one or more compounds that cause the fraction shift can include obtaining a metabolomics profile for the fraction from the second plurality of fractions; obtaining a metabolomics profile for the compound library or the natural extract; and identifying one or more compounds present in both the fraction and the compound library or the natural extract. Optionally, identifying the one or more compounds that cause the fraction shift further includes obtaining a metabolomics profile for the first sample; and filtering the metabolomics profile for the fraction from the second plurality of fractions to exclude compounds present in the first sample. Identifying the one or more compounds that cause the fraction shift may optionally include confirming co-elution of the one or more compounds and the target protein or the one or more binding proteins based on a peak elution fraction of the one or more compounds and a peak elution fraction of the target protein or the binding protein. The method may further optionally include identifying the binding protein that forms the complex with the target protein, wherein co-elution of the binding proteins with the target protein in the first plurality of fractions but not the second plurality of fractions indicates that one or more compounds in the compound library or the natural extract causes the dissociation of a complex comprising the target protein and at the binding protein.

Further described herein are systems that may be used to implement the methods described herein. Such systems may include one or more processors and a non-transitory computer readable storage medium storing one or more programs that, when executed by the one or more processors, cause the system to perform the method steps described herein. The system may further include a liquid chromatography system (which may include a size-exclusion chromatography column and/or a reverse-phase chromatography column) and/or a mass spectrometry (or tandem mass spectrometry) system.

### Definitions

As used in this specification and the appended claims, the singular forms “a”, “an” and “the” include plural referents unless the context clearly dictates otherwise. Thus, for example, reference to “a cell” optionally includes a combination of two or more such cells, and the like.

The terms “about” and “approximately” as used herein refer to the usual error range for the respective value readily known to the skilled person in this technical field. Exemplary degrees of error are within 20 percent (%), typically, within 10%, and more typically, within 5% of a given value or range of values. Reference to “about” or “approximately” a value or parameter herein includes (and describes) embodiments that are directed to that value or parameter per se.

It is understood that aspects and embodiments of the invention described herein include “comprising,” “consisting,” and “consisting essentially of” aspects and embodiments.

A “compound library” is any collection of a plurality of compounds. The compound library may include any number of compounds of 2 or more, such as 5 or more, 50 or more, 100 or more, 500 or more, or 1000 or more small molecule compounds. The compound library may be a small molecule library.

An “extract” is a biological material that has been processed to remove or substantially remove one or more components of the material. For example, an extract may be processed to remove on or more of fats, carbohydrates, or proteins. An extract may contain proteins or may be free substantially free of proteins. An extract is considered “substantially free of proteins” of the extract contains 5% (by mass) or less of its original protein content (i.e., from the natural state of the biological material).

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. A sample may be or may be an extract from a tissue, cells, sub-cellular structures (e.g., organelles), or a cell-free biological sample (e.g., saliva, plasma, cerebrospinal fluid, etc.).

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient herein is a human.

A “small molecule” is any molecule of 1000 daltons or less in molecular weight.

Where a range of values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

It is to be understood that one, some or all of the properties of the various embodiments described herein may be combined to form other embodiments of the present invention. The section headings used herein are for organizational purposes only and are not to be construed as limiting the subject matter described.

Features and preferences described above in relation to “embodiments” are distinct preferences and are not limited only to that particular embodiment; they may be freely combined with features from other embodiments, where technically feasible, and may form preferred combinations of features. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### Methods for Identifying a Protein-Protein Complex

Methods for identifying components of a protein-protein complex are described herein. Protein-protein complexes may be formed, stabilized, or dissociated in the presence of a compound (e.g., a small molecule or a drug), which may be part of a compound library or a natural extract, or a single drug tested alone. A sample, such as a biological sample (e.g., a cell-free biological sample, a tissue extract, a cellular extract, or a sub-cellular extract) can be mixed with a compound library, a drug (e.g., a drug under investigation), or a natural extract (such as plant extract). Protein complexes can be identified by fractioning (for example, by size-exclusion chromatography) the samples to obtain a plurality of fractions (i.e., a first plurality of fractions for the first sample and a second plurality of fractions for the second sample); analyzing the first and second plurality of fractions to identify proteins in the fractions (e.g., using a proteomic analysis); identifying, for a target protein, a fractionation shift between the first plurality of fractions and the second plurality of fractions; and identifying one or more binding proteins that form a complex with the target protein.

For a target protein, comparing the protein migration (e.g., elution profile) in the presence or absence of a compound library, drug or natural extract can determine whether a complex is formed or stabilized, or dissociated, by the drug or one or more compounds in the compound library or the natural extract. That is, a fraction shift caused by the compound library, drug, or natural extract, can be identified by comparing the elution of a target protein with the compound library, drug, or natural extract and without the compound library, drug, or natural extract. The fraction shift can be used to determine whether the compound library, drug, or natural extract causes the formation of a stabilizing molecular complex or a disrupting molecular complex. Binding proteins that form complexes with the target protein can also be identified by analyzing the fractions for other proteins that migrate similarly (e.g., co-elute or co-migrate) to the target protein in the presence or absence of the compound library or natural extract. Co-elution of the one or more binding proteins with the target protein in the second plurality of fractions but not the first plurality of fractions indicates that the drug or a compound in the compound library or the natural extract causes the formation or stabilization of a complex comprising the target protein and at least one of the one or more binding proteins. Co-elution of the one or more binding proteins with the target protein in the first plurality of fractions but not the second plurality of fractions indicates that the drug or a compound in the compound library or the natural extract causes the dissociation of a complex comprising the target protein and at least one of the one or more binding proteins.

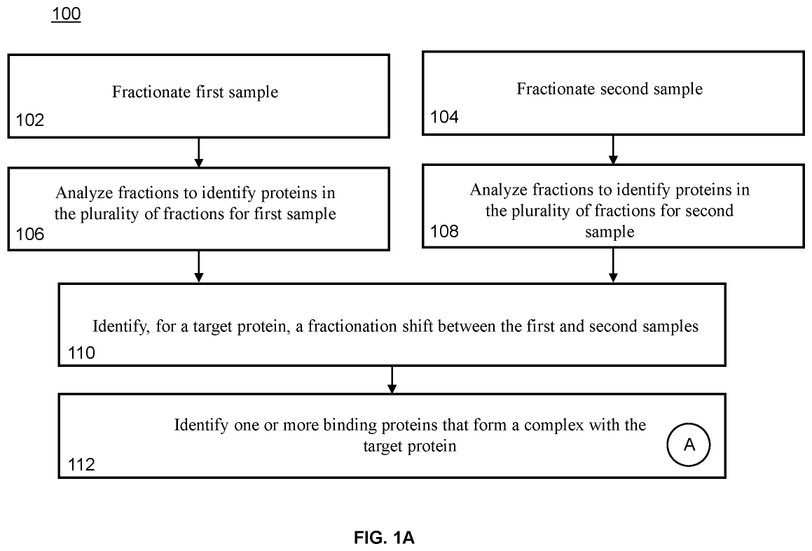

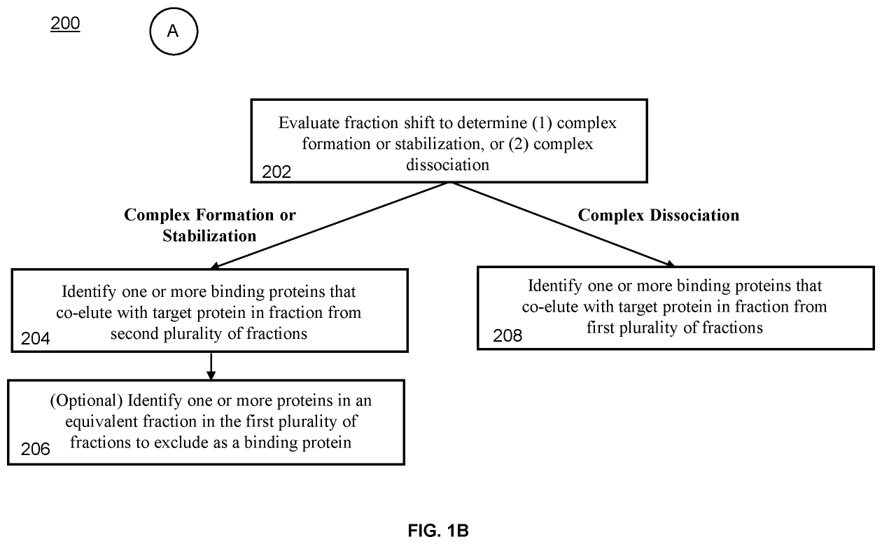

An exemplary method for identifying protein-protein complex is illustrated in FIG. 1A. A portion of a biological sample (such as a cell-free biological sample, a tissue extract, a cellular extract, or a sub-cellular extract) is first analyzed to identify the baseline elution profile of proteins and protein complexes within the sample. A drug, compound library, or a natural extract is added to another portion of the biological sample. This sample (i.e., containing the drug, compound library, or natural extract) is separately analyzed to identify the elution profile of proteins and protein complexes within the sample in the presence of the drug, compound library or natural extract. Proteins that elute in different fractions between the two samples are determined to have a fraction shift. Depending on the direction of the fraction shift, the shift can indicate that the protein either associates with or dissociates from its binding partners in the presence of the drug, compound library or natural extract. The methods described use fraction shifts to identify binding proteins (e.g., binding partners) of a target protein that associate or dissociate in the presence of the drug, compound library or natural extract.

The samples (e.g., a first sample and a second sample) are separately fractionated such as in 102 and 104 of FIG. 1A. A first sample can be a portion of a biological sample (e.g. a cell-free biological sample, a tissue extract, a cellular extract, or a sub-cellular extract). The second sample is another portion of the biological sample combined with a drug, compound library, or a natural extract (such as a plant extract). Combining may optimally include mixing or incubating the biological sample with the drug, compound library, or natural extract prior to fractioning. Incubation of the biological sample and the drug, compound library, or natural extract can occur at various temperatures and/or durations, but under conditions that preferably retain sample integrity. Incubation may also occur under physiological conditions. Incubation may be performed while the samples are non-stationary (e.g., rotation or agitation) or stationary. Optionally, one or more protease inhibitors may be added to the sample during incubation. Sample integrity can be determined by assaying for proteolysis, protein unfolding and/or protein aggregation.

The biological sample may be obtained by lysing cells from the tissue or sub-cellular structures isolated from cells. Cells, tissue, and/or sub-cellular structures may be lysed, for example, by sonication or detergent. The lysed material may be processed, for example by centrifugation and/or filtration (e.g., to remove cellular solids), by enzymes or chemicals (e.g., to remove nucleic acids), dialysis or buffer exchange, or other processing techniques. For a sub-cellular lysate, target sub-cellular structures (e.g., mitochondria, nucleus, and other organelles) may be separated from other cellular components using standard isolation techniques such as density gradient centrifugation. The cellular extract sample volume may be further reduced prior to sample fractioning. Preferably, such processing of the extract does not denature or induce proteolysis or aggregation of the proteins in the lysate. As further discussed herein, the biological sample is not limited to cellular extract, as, in some implementations, the biological sample may be a cell-free biological sample (e.g., saliva, cerebrospinal fluid, plasma, etc.) or an extract of a cell-free biological sample.

In some embodiments, the biological sample comprising proteins is obtained from animal tissue. In some embodiments, the biological sample comprising proteins is obtained from mammalian tissue. In some embodiments, the biological sample is obtained from brain, liver, lung, or kidney tissue.

The biological sample can be portioned such that a first and second portions may be used for a first sample and a second sample, respectively. The first sample comprises a portion of the biological sample comprising proteins. In some embodiments, the first sample comprises a biological sample comprising proteins that is obtained from a tissue, a cell, or a sub-cellular organelle. In some embodiments, the biological sample (e.g., tissue, cellular, or sub-cellular extract) comprising proteins is obtained from animal tissue. In some embodiments, the biological sample comprising proteins is obtained from mammalian tissue. In some embodiments, the biological sample comprising proteins is obtained from brain, liver, lung, or kidney tissue. In some embodiments, the method for identifying components of a protein-protein complex comprises fractioning a first sample to generate a first plurality of fractions. In some embodiments, the method for identifying proteins comprises fractioning a first sample by size-exclusion chromatography to generate a first plurality of fractions.

The second sample includes the portion of the biological sample that is combined with a compound library (e.g., a small-molecule library), a drug, or a natural extract (e.g., a plant, animal, bacterial, or fungal extract). The compound library may comprise fully synthetic compounds, fully natural compounds, or a mixture of synthetic and natural compounds. The natural extract may be from a different taxonomically classified organism as the organism giving rise to the biological sample. For example, the tissue, cell, or subcellular extract may be from a different kingdom (e.g., animalia, plantae, fungi, protista, archaea, or bacteria), phylum, class, order, family, genus, or species) as the origin of the natural extract. The combined components of the second sample may be mixed and/or incubated to allow components of the compound library, drug, or natural extract to interact or bind components of the biological sample. In some embodiments, the drug, compound library, or the natural extract is substantially free of proteins. Preferably, differences between the first sample and the second sample in the presence of the compound library, drug, or natural extract in the second sample.

Methods for identifying components of a protein-protein complex can include fractioning the first and second samples to generate a first plurality of fractions (i.e., for the first sample) and a second plurality of fractions (i.e., for the second sample), as shown in 102 and 104 of FIG. 1A. Samples can be fractionated to obtain a plurality of fractions using a fractioning method such as size exclusion chromatography or high-performance liquid chromatography (HPLC). Fractioning is a separation process where a sample is divided into a number of smaller quantities or fractions based on one or more physical characteristics of the components of the sample, for example hydrodynamic diameter (for example, when separating components based on size exclusion chromatography) or molecular mass. Hydrodynamic diameter is an adequate approximation for molecular weight when used in accordance with the methods described herein. Fractions are collected based on one or more differences in specific properties of the individual components. To ensure protein complexes are not disrupted during the fractionation process, fractionation of the samples should be performed in non-denaturing conditions. For example, fractionation may occur under physiological conditions, for example at a pH between about 6 and about 8. Fractioning may also occur over a range of temperatures that may or may not be physiological. In some implementations of the method described herein, fractionation occurs in phosphate buffered saline. In some embodiments, the method for identifying components of a protein-protein complex comprises fractioning samples using size-exclusion chromatography.

Fractioning the sample can be performed at a constant flow rate and/or for a set volume. The final sample is diluted into the several fractions obtained after fractioning. The fractioned samples do not contain the same proteins and/or compounds across all fractions because they will be separated based on one or more criteria selected for by the fractioning technique. For example, size exclusion chromatography separates a mixture based on physical properties such as size and shape (hydrodynamic size) of the protein or protein complex. The fractions may further comprise one or more compounds.

Fractions are collected and analyzed to identify the proteins in each fraction. As shown at 106 of FIG. 1A, fractions from the first sample are analyzed to identify proteins in the plurality of fractions for the first sample, and, as shown at 108, fractions from the second sample are analyzed to identify proteins in the plurality of fractions for the second sample. This identification may be performed, for example, using a proteomics analysis. Exemplary proteomics analytical techniques can include the use of mass spectrometry (e.g., liquid chromatography with tandem mass spectrometry (LC-MS/MS). Prior to identification, samples may be further processed. For example, samples may also be separated by SDS-PAGE and proteins of a specific size may be excised out of the gel for further analysis. Liquid or gel-based samples may also be deliberately digested using one or more proteases to fragment proteins into shorter polypeptides prior to protein identification. In some embodiments, the protease is an amino acid specific protease. In some embodiments, the protease cuts only at the N-terminus of an amino acid. In some embodiments, the protease cuts only at the C-terminus of an amino acid. The fractions may also be subject to further processing to prepare the samples such as buffer exchange to remove salts and other buffer components that may interfere with analysis. For example, to prepare the samples for LC-MS/MS, the proteins in the fractions are precipitated out of solution using kits (comprising components such as buffers) or chemicals, then resuspended in a compatible buffer. Analysis of the samples may be performed using protein identification techniques including western blotting or LC-MS/MS. The collected fractions may be further separated by using liquid chromatography to separate peptides, then the eluate is directed towards a mass spectrometer with an ionization source. Methods useful for identifying proteins in the fractions include proteomic methods such as immunoassays, mass spectrometry and/or combinations thereof. In some embodiments, the identifying proteins in the first and second plurality of fractions comprise the use of mass spectrometry. In some embodiments, the mass spectrometry is liquid-chromatography mass spectrometry (LC-MS/MS). In some embodiments, the methods for identifying proteins in the first fraction and the second fraction are the same method.

A protein of interest may be identified within the biological sample. The protein of interest may be, for example, a drug target or potential drug target. This protein can be designated a target protein. A target protein that elutes in a different fraction (e.g., has a fraction shift) in the presence of a drug, a compound library, or a natural extract indicates that it has experienced a change in complex status (e.g., through complex formation or stabilization, or dissociation), as evidenced by the change in hydrodynamic size. For example, binding proteins co-elute with the target protein because the complex remains associated during fractioning. At 110 of FIG. 1A, a fractionation shift for the target protein between the first and second samples can be identified. Identifying a fraction shift for the target protein indicates that the target protein is interacting differently with other proteins (e.g., binding proteins) in the presence or absence of a drug, a compound library, or a natural extract, for example by complex formation or stabilization, or complex dissociation.

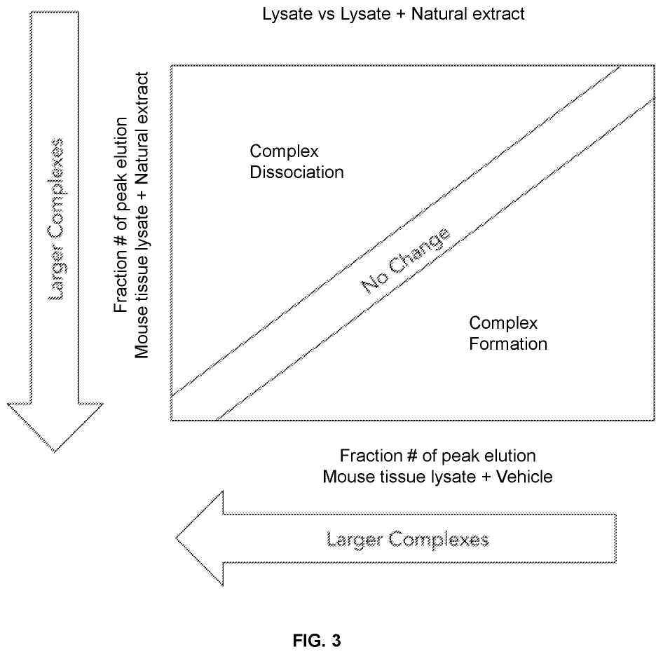

Identifying a fraction shift may include converting the fractionation information and protein identity data into a two-dimensional matrix, for example as shown in FIG. 3. On one axis, the fraction numbers corresponding to one sample (e.g., the first sample) is presented. On the other axis, the fraction numbers corresponding to another sample (e.g., the second sample) is presented. Datapoints representing the identified proteins are assigned coordinates on the plot based on the fractions in which they elute in either sample. While protein elution is a distribution and may cover a range of fractions, the peak elution fraction is assigned based on the greatest abundance of protein observed between all fractions (e.g., maximum intensity).

Evaluation of the fraction shift for a protein, such as a target protein, can indicate whether the drug or a compound in the compound library or natural extract causes complex formation or stabilization, or dissociation. If the protein elutes in a different fraction in one sample but not the other sample, the data point that represents the protein will be located off the diagonal (FIG. 3). If a target protein elutes in a later fraction in the first sample (biological sample without the drug, compound library, or natural extract) than the protein elutes in the second sample (biological sample with the drug, compound library, or natural extract), it can be concluded that the drug, compound library, or natural extract causes the formation or stabilization of a complex comprising the target protein. This is because the drug, compound library, or natural extract causes the target protein to associate in a species with a higher molecular weight. In contrast, if a target protein elutes in an earlier fraction in the first sample (biological sample without the drug, compound library, or natural extract) than the protein elutes in the second sample (biological sample with the drug, compound library, or natural extract), it can be concluded that the drug, compound library, or natural extract causes the dissociation of a complex comprising the target protein. If a protein does not change its peak elution fraction between both samples, the data point representing the protein will be located along a diagonal (e.g., be assigned to the same fraction in both samples).

At 108 of the method shown in FIG. 1A, one or more binding protein that form a complex with the target protein (either in the first sample or the second sample) are identified. An exemplary process for identifying the one or more binding proteins is shown in further detail in FIG. 1B. At 202, the fraction shift is evaluated to determine whether the drug, compound library, or natural extract causes complex formation or stabilization, or complex dissociation. Co-elution of one or more additional proteins with the target protein in either the first sample or the second sample, but not both, indicates that the drug or one or more compounds in the compound library or natural extract causes formation or stabilization, or complex dissociation, of a complex that includes the target protein and one or more of the additional proteins. Thus, the one or more proteins (in addition to the target protein) in the complex may be referred to as “binding proteins” as they are involved in a target-protein containing complex either in the first sample or the second sample. Proteins that co-elute in the same fraction as the target protein in the presence of the drug, compound library, or natural extract (but do not co-elute without the drug, compound library, or natural extract) indicate that the additional one or more protein(s) are binding proteins that form a complex with the target protein in the presence of the drug, compound library, or natural extract. Proteins that co-elute in the same fraction as the target protein without the presence of the drug, compound library, or natural extract (but do not co-elute in the presence of the drug, compound library, or natural extract) indicate that the additional one or more protein(s) are binding proteins form a complex with the target protein in the absence of the drug, compound, library, or natural extract. If the drug, compound library, or natural extract causes complex formation or stabilization, proteins that co-elute with the target protein in the second plurality fractions (i.e., the plurality of fractions for the second sample that includes the biological sample and the drug, compound library, or natural extract) can be identified as binding proteins for the target protein, as shown at 204. Optionally, the equivalent fraction (e.g., the same fraction number) for the first plurality of fractions (i.e., the plurality of fractions for the first sample that includes the biological sample and does not include the drug, compound library, or natural extract) can be analyzed, with proteins present in said fraction being excluded as binding proteins, as shown at 206. If the drug, compound library, or natural extract causes complex dissociation, proteins that co-elute with the target protein in the first plurality of fractions (i.e., the plurality of fractions for the first sample that includes the biological sample and does not include the drug, compound library, or natural extract) can be identified as binding proteins of the target protein, as shown in 208.

### Methods for Identifying a Compound that Modulates a Protein Complex

Methods for identifying components of a compound that modulates a protein complex are described herein. Protein-protein complexes may be formed or stabilized, or dissociated, in the presence of one or more compounds, for example one or more compounds from a compound library or a natural extract. A sample such as a biological sample (e.g., a cell-free biological sample, a tissue extract, a cellular extract, or a sub-cellular extract) can be mixed with a compound library or a natural extract (such as plant extract). Protein complexes can be identified by fractioning samples to obtain a plurality of fractions. Fractioning samples can be done by a variety of methods, for example size exclusion chromatography. Proteomic analysis can be applied to the fractions to identify the proteins. Metabolomic analysis (such as LC-MS/MS or in silico NMR) can also be applied to identify one or more compounds to the fractions. The combination of proteomic and metabolomic analysis may be used to identify one or more compounds that cause complex formation or stabilization, or dissociation.

For example, a compound may cause a target protein to associate with one or more binding partners. In some implementations, the compound will remain bound to the newly formed protein complex as the sample is fractioned. Therefore, identifying one or more compounds that co-elute with the target protein and its binding partners indicates the compound modulates protein complex formation. Identifying the one or more compounds that cause the fraction shift may be done by obtaining metabolomics profiles, for example, by tandem mass spectrometry (LC-MS/MS) for the fractions, and identifying compounds present in both the fraction and the compound library or natural extract. In some implementations, a metabolomics profile of the fractions for the first sample (e.g., a first fraction) is compared to the metabolomics profile of the fraction from the second sample. For example, an increased amount of a compound in the second fraction (e.g., a second fraction) when compared to first sample indicates a new co-eluting compound. Co-elution may be determined based on the fraction where the peak elution (e.g., greatest abundance, maximum intensity) of the protein and/or compound is detected.

In some implementations compound may cause a target protein to dissociate with its one or more binding partners. For example, the compound may remain bound to either the target protein or another member of the complex (e.g., one or more binding partners) as the sample is fractioned. Therefore, identifying one or more compounds that co-elute with the target protein or its binding partners indicates the compound modulates protein complex dissociation. Identifying the one or more compounds that cause the fraction shift may be done by obtaining metabolomics profiles, for example, by tandem mass spectrometry (LC-MS/MS) for the fractions, and identifying compounds present in both the fraction and the compound library or natural extract. Another method to determine whether the compound is co-eluting in a new fraction, a metabolomics profile of the fractions for the first sample (e.g., a first fraction) is compared to the metabolomics profile of the fraction from the second sample. For example, enrichment of a compound in the second fraction (e.g., a second fraction) when compared to first sample indicates a new co-eluting compound. Co-elution may be defined by the fraction where the peak elution (e.g., greatest abundance, maximum intensity) of the protein and/or compound is detected.

For a target protein, comparing the protein migration (e.g., elution profile) in the presence or absence of a compound library or natural extract can determine whether a complex is formed or stabilized, or dissociated, by the one or more compounds in the compound library or the natural extract. That is, a fraction shift caused by the compound library or natural extract, can be identified by comparing the elution of a target protein with the compound library or natural extract and without the compound library or natural extract. The fraction shift can be used to determine whether the compound library or natural extract causes the formation of a stabilizing molecular complex or a disrupting molecular complex. Binding proteins that form complexes with the target protein can also be identified by analyzing the fractions for other proteins that migrate similarly (e.g., co-elute or co-migrate) to the target protein in the presence or absence of the compound library or natural extract. Co-elution of the one or more binding proteins with the target protein in the second plurality of fractions but not the first plurality of fractions indicates that the drug or a compound in the compound library or the natural extract causes the formation or stabilization of a complex comprising the target protein and at least one of the one or more binding proteins. Co-elution of the one or more binding proteins with the target protein in the first plurality of fractions but not the second plurality of fractions indicates that the drug or a compound in the compound library or the natural extract causes the dissociation of a complex comprising the target protein and at least one of the one or more binding proteins.

The compound responsible for causing the complex formation or stabilization, or complex dissociation, is expected to co-elute with the target proteins and/or the one or more binding proteins. For example, if the compound is responsible for complex formation or stabilization, the compound would be expected to bind to the target protein and/or binding protein, and therefore co-elute with the target protein and the one or more binding proteins in the plurality of fractions for the sample containing the biological sample and the compound library or natural extract. If the compound is responsible for complex dissociation, the compound would be expected to bind to either the target protein or the binding protein, and therefore co-elute with the target protein or the binding protein in the plurality of fractions for the sample containing the biological sample and the compound library or natural extract.

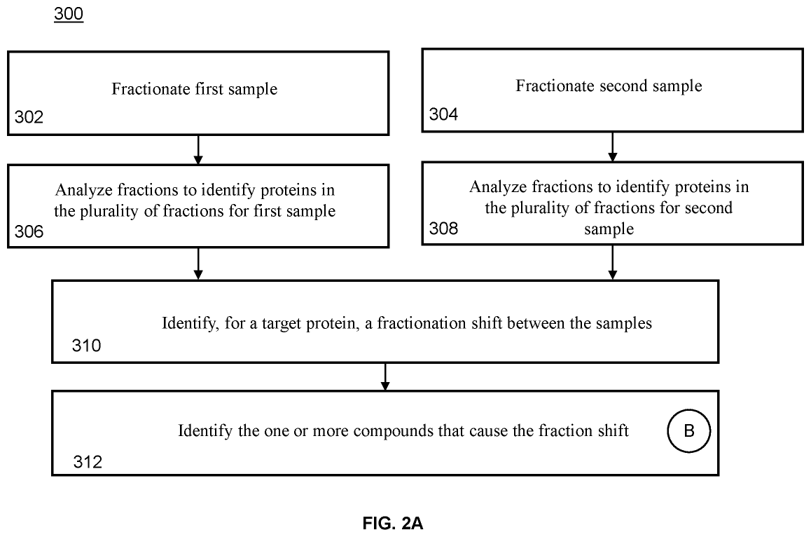

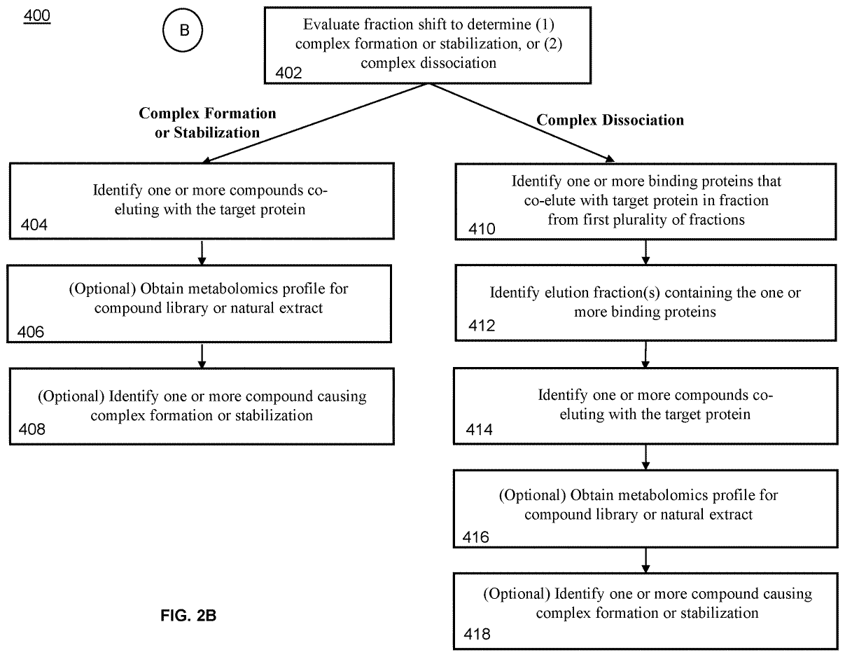

An exemplary method for identifying protein-protein complex is illustrated in FIG. 2A. A portion of a biological sample (such as a cell-free biological sample, a tissue extract, a cellular extract, or a sub-cellular extract) is first analyzed to identify the baseline elution profile of proteins and protein complexes within the sample. A compound library or a natural extract is added to another portion of the biological sample. This sample (i.e., containing the drug, compound library, or natural extract) is separately analyzed to identify the elution profile of proteins and protein complexes within the sample in the presence of the drug, compound library or natural extract. Proteins that elute in different fractions between the two samples are determined to have a fraction shift. Depending on the direction of the fraction shift, the shift can indicate that the protein either associates with or dissociates from its binding partners in the presence of the compound library or natural extract.

The samples (e.g., a first sample and a second sample) are separately fractionated such as in 302 and 304 of FIG. 2A. A first sample can be a portion of a biological sample (e.g., a cell-free biological sample, a tissue extract, a cellular extract, or a sub-cellular extract). The second sample is another portion of the biological sample combined with a compound library or a natural extract (such as a plant extract). Combining may optimally include mixing or incubating the biological sample with the compound library or natural extract prior to fractioning. Incubation of the biological sample and the compound library or natural extract can occur at various temperatures and/or durations, but under conditions that preferably retain sample integrity. Incubation may also occur under physiological conditions. Incubation may be performed while the samples are non-stationary (e.g., rotation or agitation) or stationary. Optionally, one or more protease inhibitors may be added to the sample during incubation. Sample integrity can be determined by assaying for proteolysis, protein unfolding and/or protein aggregation.

The biological sample may be obtained by lysing cells from the tissue or sub-cellular structures isolated from cells. Cells, tissue, and/or sub-cellular structures may be lysed, for example, by sonication or detergent. The lysed material may be processed, for example by centrifugation and/or filtration (e.g., to remove cellular solids), by enzymes or chemicals (e.g., to remove nucleic acids), dialysis or buffer exchange, or other processing techniques. For a sub-cellular lysate, target sub-cellular structures (e.g., mitochondria, nucleus, and other organelles) may be separated from other cellular components using standard isolation techniques such as density gradient centrifugation. The cellular extract sample volume may be further reduced prior to sample fractioning. Preferably, such processing of the extract does not denature or induce proteolysis or aggregation of the proteins in the lysate. As further discussed herein, the biological sample is not limited to cellular extract, as, in some implementations, the biological sample may be a cell-free biological sample (e.g., saliva, cerebrospinal fluid, plasma, etc.) or an extract of a cell-free biological sample.

In some embodiments, the biological sample comprising proteins is obtained from animal tissue. In some embodiments, the biological sample comprising proteins is obtained from mammalian tissue. In some embodiments, the biological sample is obtained from brain, liver, lung, or kidney tissue.

The biological sample can be portioned such that a first and second portions may be used for a first sample and a second sample, respectively. The first sample comprises a portion of the biological sample comprising proteins. In some embodiments, the first sample comprises a biological sample comprising proteins that is obtained from a tissue, a cell, or a sub-cellular organelle. In some embodiments, the biological sample (e.g., tissue, cellular, or sub-cellular extract) comprising proteins is obtained from animal tissue. In some embodiments, the biological sample comprising proteins is obtained from mammalian tissue. In some embodiments, the biological sample comprising proteins is obtained from brain, liver, lung, or kidney tissue. In some embodiments, the method for identifying components of a protein-protein complex comprises fractioning a first sample to generate a first plurality of fractions. In some embodiments, the method for identifying proteins comprises fractioning a first sample by size-exclusion chromatography to generate a first plurality of fractions.

The second sample includes the portion of the biological sample that is combined with a compound library (e.g., a small-molecule library) or a natural extract (e.g., a plant, animal, bacterial, or fungal extract). The compound library may comprise fully synthetic compounds, fully natural compounds, or a mixture of synthetic and natural compounds. The natural extract may be from a different taxonomically classified organism as the organism giving rise to the biological sample. For example, the tissue, cell, or subcellular extract may be from a different kingdom (e.g., animalia, plantae, fungi, protista, archaea, or bacteria), phylum, class, order, family, genus, or species) as the origin of the natural extract. The combined components of the second sample may be mixed and/or incubated to allow components of the compound library, drug, or natural extract to interact or bind components of the biological sample. In some embodiments, compound library, drug, or the natural extract is substantially free of proteins. Preferably, differences between the first sample and the second sample in the presence of the compound library or natural extract in the second sample.

Methods for identifying a compound that modulates complex formation, stabilization, or dissociation can include fractioning the first and second samples to generate a first plurality of fractions (i.e., for the first sample) and a second plurality of fractions (i.e., for the second sample), as shown in 302 and 304 of FIG. 2A. Samples can be fractionated to obtain a plurality of fractions using a fractioning method such as size exclusion chromatography or high-performance liquid chromatography (HPLC). Fractioning is a separation process where a sample is divided into a number of smaller quantities or fractions based on one or more physical characteristics of the components of the sample, for example hydrodynamic diameter (for example, when separating components based on size exclusion chromatography) or molecular mass. Hydrodynamic diameter is an adequate approximation for molecular weight when used in accordance with the methods described herein. Fractions are collected based on one or more differences in specific properties of the individual components. To ensure protein complexes are not disrupted during the fractionation process, fractionation of the samples should be performed in non-denaturing conditions. For example, fractionation may occur under physiological conditions, for example at a pH between about 6 and about 8. Fractioning may also occur over a range of temperatures that may or may not be physiological. In some implementations of the method described herein, fractionation occurs in phosphate buffered saline. In some embodiments, the method for identifying components of a protein-protein complex comprises fractioning samples using size-exclusion chromatography.

Fractioning the sample can be performed at a constant flow rate and/or for a set volume. The final sample is diluted into the several fractions obtained after fractioning. The fractioned samples do not contain the same proteins and/or compounds across all fractions because they will be separated based on one or more criteria selected for by the fractioning technique. For example, size exclusion chromatography separates a mixture based on physical properties such as size and shape (hydrodynamic size) of the protein or protein complex. The fractions may further comprise one or more compounds.

Fractions are collected and analyzed to identify the proteins in each fraction. As shown at 306 of FIG. 1A, fractions from the first sample are analyzed to identify proteins in the plurality of fractions for the first sample, and, as shown at 308, fractions from the second sample are analyzed to identify proteins in the plurality of fractions for the second sample. This identification may be performed, for example, using a proteomics analysis. Exemplary proteomics analytical techniques can include the use of mass spectrometry (e.g., liquid chromatography with tandem mass spectrometry (LC-MS/MS). Prior to identification, samples may be further processed. For example, samples may also be separated by SDS-PAGE and proteins of a specific size may be excised out of the gel for further analysis. Liquid or gel-based samples may also be deliberately digested using one or more proteases to fragment proteins into shorter polypeptides prior to protein identification. In some embodiments, the protease is an amino acid specific protease. In some embodiments, the protease cuts only at the N-terminus of an amino acid. In some embodiments, the protease cuts only at the C-terminus of an amino acid. The fractions may also be subject to further processing to prepare the samples such as buffer exchange to remove salts and other buffer components that may interfere with analysis. For example, to prepare the samples for LC-MS/MS, the proteins in the fractions are precipitated out of solution using kits (comprising components such as buffers) or chemicals, then resuspended in a compatible buffer. Analysis of the samples may be performed using protein identification techniques including western blotting or LC-MS/MS. The collected fractions may be further separated by using liquid chromatography to separate peptides, then the eluate is directed towards a mass spectrometer with an ionization source. Methods useful for identifying proteins in the fractions include proteomic methods such as immunoassays, mass spectrometry and/or combinations thereof. In some embodiments, the identifying proteins in the first and second plurality of fractions comprise the use of mass spectrometry. In some embodiments, the mass spectrometry is liquid-chromatography mass spectrometry (LC-MS/MS). In some embodiments, the methods for identifying proteins in the first fraction and the second fraction are the same method.

A protein of interest may be identified within the biological sample. The protein of interest may be, for example, a drug target or potential drug target. This protein can be designated a target protein. A target protein that elutes in a different fraction (e.g., has a fraction shift) in the presence of a compound library or a natural extract indicates that it has experienced a change in complex status (e.g., through complex formation or stabilization, or dissociation), as evidenced by the change in hydrodynamic size. For example, binding proteins co-elute with the target protein because the complex remains associated during fractioning. At 310 of FIG. 2A, a fractionation shift for the target protein between the first and second samples can be identified. Identifying a fraction shift for the target protein indicates that the target protein is interacting differently with other proteins (e.g., binding proteins) in the presence or absence of a drug, a compound library, or a natural extract, for example by complex formation or stabilization, or complex dissociation.

Identifying a fraction shift may include converting the fractionation information and protein identity data into a two-dimensional matrix, for example as shown in FIG. 3. On one axis, the fraction numbers corresponding to one sample (e.g., the first sample) is presented. On the other axis, the fraction numbers corresponding to another sample (e.g., the second sample) is presented. Datapoints representing the identified proteins are assigned coordinates on the plot based on the fractions in which they elute in either sample. While protein elution is a distribution and may cover a range of fractions, the peak elution fraction is assigned based on the greatest abundance of protein observed between all fractions (e.g., maximum intensity).

Evaluation of the fraction shift for a protein, such as a target protein, can indicate whether the drug or a compound in the compound library or natural extract causes complex formation or stabilization, or dissociation. If the protein elutes in a different fraction in one sample but not the other sample, the data point that represents the protein will be located off the diagonal (FIG. 3). If a target protein elutes in a later fraction in the first sample (biological sample without the compound library or natural extract) than the protein elutes in the second sample (biological sample with the compound library or natural extract), it can be concluded that the compound library or natural extract causes the formation or stabilization of a complex comprising the target protein. This is because the compound library or natural extract causes the target protein to associate in a species with a higher molecular weight. In contrast, if a target protein elutes in an earlier fraction in the first sample (biological sample without the compound library or natural extract) than the protein elutes in the second sample (biological sample with the compound library or natural extract), it can be concluded that the compound library, or natural extract causes the dissociation of a complex comprising the target protein. If a protein does not change its peak elution fraction between both samples, the data point representing the protein will be located along a diagonal (e.g., be assigned to the same fraction in both samples).

At 308 of FIG. 2A, one or more compounds that cause the fraction shift (i.e., that cause complex formation or stabilization, or complex dissociation) are identified. Identification of the one or more compounds may include a metabolomics analysis of the fraction from the second plurality of fractions containing the target protein and/or a binding protein. The process for identifying the one or more compounds that cause the fraction shift may differ depending on whether the compound library or the natural extract causes complex formation or stabilization, or complex dissociation.

An exemplary process for identifying the one or more compounds is shown in FIG. 2B. At 402 of FIG. 2B, the fraction shift is evaluated to determine whether the compound library or natural extract causes complex formation or stabilization, or complex dissociation. If the fraction shift indicates complex formation or stabilization, one or more compounds co-eluting with the target protein are identified at 404, for example by obtaining a metabolomics profile for the elution fraction comprising the target protein. In some implementations, the co-elution is based on a fraction containing peak elution of the target protein and the compound. For example, a metabolomics and proteomics analysis of the target protein elution fraction and one or more adjacent fractions may be performed to determine the elution fraction of the compound and the target protein. Optionally, a metabolomics profile of the compound library or natural extract may be obtained (for example, by performing a metabolomics assay on the compound library or natural extract), as shown in 406. The metabolomics profile of the compound library or natural extract may be used to identify the compound that co-elutes with the target protein. Optionally, a metabolomics profile of the biological sample may be obtained (for example, by performing a metabolomics assay on the biological sample). The metabolomics profile of the biological sample may be used to exclude a compound as causing the complex formation or stabilization. The one or more compounds that cause the fraction shift may be confirmed by identifying one or more compounds present both in the fraction comprising the target protein and in the compound library or the natural extract, as shown in 408.

If the fraction shift indicates complex dissociation, the one or more compounds causing complex dissociation may be bound to (and co-elute with) either the target protein or one or more binding proteins. Thus, if the fraction shift indicates complex dissociation, one or more binding proteins (i.e., one or more proteins that for a complex with the target protein in the absence of the one or more compounds from the compound library or natural extract) may be identified, as shown at 410. As discussed above, proteins that co-elute in the same fraction as the target protein without the compound library or natural extract (but do not co-elute in the presence of the compound library or natural extract) indicate that the additional one or more protein(s) are binding proteins form a complex with the target protein in the presence of the compound, library or natural extract. Thus, the one or more binding proteins may be identified by identifying one or more proteins that co-elute with the target protein in a fraction from the first plurality of fractions, for example by performing a proteomics analysis on one or more fractions from the first plurality of fractions (i.e., the fractions associated with the sample comprising the biological sample without the natural extract or compound library). Once one or more binding proteins have been identified, the elution fraction (or elution fractions) for the one or more binding proteins from the second plurality of fractions (i.e., the factions associated with the sample comprising the biological sample and the compound library or natural extract) can identified, as shown at 412, for example by a proteomics analysis of the fractions in the second plurality of fractions. One or more compounds co-eluting with the one or more binding proteins, or with the target protein, may then be identified at 414, for example using a metabolomics analysis. In some implementations, the co-elution is based on an elution fraction containing peak elution of the target protein or binding protein and the compound. For example, a metabolomics and proteomics analysis of the target protein elution fraction and/or binding protein elution fraction and one or more adjacent fractions may be performed to determine the elution fraction of the compound and the target protein or binding protein. Optionally, a metabolomics profile of the compound library or natural extract may be obtained (for example, by performing a metabolomics assay on the compound library or natural extract), as shown in 416. The metabolomics profile of the compound library or natural extract may be used to identify the compound that co-elutes with the target protein. Optionally, a metabolomics profile of the biological sample may be obtained (for example, by performing a metabolomics assay on the biological sample). The metabolomics profile of the biological sample may be used to exclude a compound as causing the complex formation or stabilization. The one or more compounds that cause the fraction shift may be confirmed by identifying one or more compounds present both in the fraction comprising the target protein (or binding protein) and in the compound library or the natural extract, as shown in 418.

### Samples

A sample (e.g., a first sample) as described herein can be a biological sample (e.g., a tissue, cellular or sub-cellular extract, or a cell-free biological sample). The biological sample may be portioned into a first sample and a second sample. The first sample can include the biological sample, but should not include the drug, compound library, or natural extract investigated according to the methods described herein. The second sample includes the same biological sample (i.e., the second portion of the biological sample), and also comprises at least one drug, a compound library, or a natural extract. The second sample is combined (for example, by mixing) with the drug, a compound library, or a natural extract. Preferably, the first and second samples differ only in the presence or absence of a drug, compound library or natural extract. A sample may be protein and polypeptides suspended in a buffer. A sample may have proteins, polypeptides, and compounds.

In some embodiments, the sample comprise proteins. In some embodiments, the sample comprises compounds. In some embodiments, the sample comprises both proteins and compounds. In some embodiments, the samples may comprise extracts from the different sources. In some embodiments, the natural extract is a plant extract.

In some embodiments, the samples may be fractioned by any of the methods disclosed herein. In some embodiments, the samples are fractioned by size exclusion chromatography. In some embodiments, the samples are fractioned to obtain a plurality of fractions. Fractioning is a separation process where a mixture is divided into a number of smaller quantities or fractions, in which the composition varies. Fractions are collected based on one or more differences in specific properties of the individual components. In some embodiments, a sample may be fractioned to obtain a plurality of fractions. In some embodiments, the plurality of fractions are of same, similar or equivalent volume. In some embodiments, the plurality of fractions may be further analyzed by proteomic and/or metabolomic methods. In some embodiments, individual fractions are collected then analyzed. In some embodiments, individual fractions are collected, pooled, then analyzed. In some embodiments, individual fractions are collected, further divided, then analyzed. In some embodiments, individual fractions are collected, further divided, then analyzed using two different methods. The following describes exemplary types of samples that are useful in the present invention.

### Biological Samples

Biological samples may be derived from or obtained from biological materials isolated from any organism, such as humans or rodents. Biological samples may from tissue extracts, cellular extracts, or sub-cellular extracts (e.g., organelle extracts), or cell-free biological samples. Exemplary cell-free biological samples include, but are not limited to, plasma samples, cerebrospinal samples, saliva samples, milk samples, sputum samples, and fecal samples. Tissues may be isolated from the organism, digested mechanically, enzymatically, or both to release cells. Cells can then be applied to further lysis protocols to create a cell extract. Prior to cell lysis, particular cell types may be isolated or sorted to obtain specific cell-type extracts. Sub-cellular extracts may also be obtained from first gently lysing cells, then applying a range of centrifugation or purification (such as tag-purification) methods to isolate in-tact organelles of interest prior to total lysis to obtain sub-cellular extracts.

Several methods are commonly used to extract proteins, including mechanical disruption, liquid homogenization, high frequency sound waves (sonication), freeze/thaw cycles, and manual grinding. The choice of cell lysis method depends on the starting material, volume, and sensitivity of proteins being extracted.

Physical disruption is an efficient method to lyse a wide range of cells and has a high lysing efficiency. Methods of physical extraction include but are not limited to any of the following or combination of the following: dounce homogenizers, sonicators, blenders, mortar and pestle, freezing with reagents such as dry ice with ethanol or liquid nitrogen, and French press. In physical disruption methods, the material is physically broken down by shear or external forces to release cellular components.

Detergents solubilize proteins and disrupting lipid-lipid, protein-protein, and protein-lipid interactions. It can be used to extract total protein or subcellular fractions or organelles from various sample types. Detergent based lysis is easily adaptable for small volumes or larger samples and is a milder alternative to physical disruption of cell membranes, although it is often used in conjunction with homogenization and mechanical grinding when preparing protein samples from tissues to achieve complete cell disruption.

In some embodiments, the methods disclosed herein fractioning a first sample. In some embodiments, in some embodiments, the first sample comprises a portion of cellular extract comprising proteins. In some embodiments, the first sample is a cellular extract comprising proteins that is obtained from a tissue, cellular, or subcellular extract. In some embodiments, the cellular extract comprising proteins is obtained from animal tissue. In some embodiments, the cellular extract comprising proteins is obtained from mammalian tissue. In some embodiments, the extract comprising proteins is obtained from brain, liver, lung, or kidney tissue.

### Natural Extracts

Extracts are mixtures of secondary metabolites. Diverse classes of compounds are found in plants and their extracts; however, most of the bioactive compounds come from four major classes: alkaloids, glycosides, polyphenols, and terpenes. Various traditional and modern methods are used to prepare the plant extract from different parts of the plants such as Soxhlet extraction, reflux extraction, sonification, decoction, maceration, pressurized-liquid extraction, solid-phase extraction, microwave-assisted extraction, hydro distillation, and enzyme-assisted extraction. Sample preparation first decomposes the matrix, then isolates the target analytes.

In some embodiments, the methods disclosed herein fractioning a second sample. In some embodiments, the second sample comprises a natural extract. In some embodiments, the second sample comprises (i) a second portion of the tissue, cellular or subcellular extract and (ii) a natural extract. In some embodiments, the natural extract is a plant extract. In some embodiments, the natural extract is substantially free of proteins.

### Compound Libraries

A chemical library or compound library is a collection of stored chemicals usually used ultimately in high-throughput screening or industrial manufacture. The chemical library can consist in simple terms of a series of stored chemicals. Each chemical has associated information stored in a database with information such as the chemical structure, purity, quantity, and physiochemical characteristics of the compound. In the drug discovery process for instance, a wide range of organic chemicals are needed to test against models of disease in high-throughput screening. A chemical library may comprise fully synthetic compounds, fully natural compounds, or a mixture of synthetic and natural compounds. The compound library may include one or more drugs and/or one or more natural extracts.

In some embodiments, the methods disclosed herein comprise fractioning a second sample comprising a natural extract. In some embodiments, the second sample comprises (i) a second portion of the cellular extract and (ii) a compound library. In some embodiments, the compound library is substantially free of proteins.

### Drug

A drug may be a chemical substance or compound of fully natural, fully synthetic or semisynthetic origin. It may be isolated from natural sources (e.g., a plant extract) as described above. A drug may also be synthesized or synthesized in part.

In some embodiments, the methods disclosed herein comprise fractioning a second sample comprising a drug. In some embodiments, the second sample comprises (i) a second portion of the cellular extract and (ii) a drug. In some embodiments, the drug is substantially free of proteins.

### Analytical Methods

The present methods utilize on or more analytical methods to separate and/or analyze a complex mixture of proteins, peptides, and compounds.

### Liquid Chromatography

One method of separating a sample into simplified or individual parts is liquid chromatography. Liquid chromatography comprises a mobile phase and a stationary phase. Samples with proteins and/or to separate a sample into its individual parts. This separation occurs based on the components of the sample with the mobile and stationary phases. The mobile phase (liquid phase) may be a buffer, preferably a physiologically relevant buffer. Liquid chromatography uses pumps to flow a pressurized liquid and sample mixture through a column filled with adsorbent (stationary phase), which separates the sample components based on their interactions with the stationary phase. In some embodiments, the buffer may be at pH 6-8. In some embodiments, the buffer comprises phosphate buffered saline. The stationary phase comprises a resin that forms a matrix. Molecules will enter the column and interact with the stationary phase differently based on their various properties. For example, in ion exchange chromatography, stationary phase may be charged to separate proteins or polypeptides based on their charge or isoelectric point.

Stationary phases may also be made of different resins to form matrices that retain proteins based on hydrodynamic size or molecular weight. Size exclusion chromatography (SEC) is a chromatographic method in which molecules (such as proteins and polypeptides) in solution are separated by their size, and in some cases molecular weight. Size exclusion columns may be selected based on the resolution of apparent molecular weights over a given range of molecular weights. In some embodiments, the samples are fractioned by size exclusion chromatography. As proteins exit the column, one or more detectors may be used to identify the presence of a protein versus buffer alone.

High performance liquid chromatography (HPLC) high-pressure liquid chromatography is similar to SEC in that it uses a stationary phase and a mobile phase to separate, identify and quantify components in a mixture. The technique is applied in analytical chemistry, and also relies on pumps to pass the sample through the column. The components within the sample will interact differentially with the stationary phase, causing them to elute at different times. The mobile phase may be a mixture of solvents (e.g., water, acetonitrile and/or methanol).

HPLC is distinguished from size exclusion chromatography because it operates under high pressure, shorter column lengths and smaller resin to yield high resolution separation of compounds. As the compounds exit the column, one or more detectors may be used to identify the presence of compound versus buffer alone. In some embodiments, samples comprising compounds are fractioned by HPLC.

### Proteomics

Proteomics is the large-scale study of proteins. As applied in the present application, proteomics analysis comprise identification of all the proteins within the sample or within each fraction, such as the plurality of fractions obtained from the fractioned sample.

Mass spectrometry (MS)-based proteomics (e.g., measuring and analyzing the quality and quantity of proteins in a sample or a fraction) may be utilized to analyze a known protein or peptide to calculate the likely fragmentation of the molecule or compound. Proteins and/or peptides in the samples of the present invention can be identified by comparison of retention time/index (IR), mass-to-charge ratio (m/z) of the ion, and MS fragmentation pattern to known proteins and/or peptides.

In some embodiments, the first plurality of fractions and the second plurality of fractions are analyzed to identify proteins in the first plurality of fractions and the second plurality of fractions. In some embodiments, the analysis comprises a proteomics analysis. In some embodiments, the analysis comprises using mass spectrometry. In some embodiments, the analysis comprises using liquid chromatography with tandem mass spectrometry (LC-MS/MS).

### Metabolomics

Metabolomics methods may be used in accordance with the described methods, for example to identify a drug, compound, or a small molecule in the sample.

### MS-Based Metabolomics

MS-based metabolomics (e.g., measuring and analyzing the quality, identity and quantity of compound in a sample or a fraction) may be utilized to analyze a known chemical structure of a compound to calculate the likely fragmentation of the compound. Compounds in the samples of the present invention can be identified by comparison of retention time/index (IR), mass-to-charge ratio (m/z) of the ion, and MS fragmentation pattern to known compounds such as those found in a compound library or native extract.

In some embodiments, identifying the one or more compounds that cause the fraction shift comprises obtaining a metabolomics profile for the fractions. In some embodiments, identifying the one or more compounds that cause the fraction shift comprises obtaining a metabolomics profile from the second plurality of fractions. In some embodiments, identifying the one or more compounds that cause the fraction shift comprises obtaining a metabolomics profile for the compound library or the natural extract. In some embodiments, identifying the one or more compounds that cause the fraction shift comprises identifying one or more compounds present in both the fraction and the compound library or the natural extract. In some embodiments, identifying the one or more compounds that cause the fraction shift comprises: (i) obtaining a metabolomics profile for the fraction from the second plurality of fractions; (ii) obtaining a metabolomics profile for the compound library or the natural extract; and (iii) identifying one or more compounds present in both the fraction and the compound library or the natural extract. In some embodiments, identifying the one or more compounds that cause the fraction shift further comprises obtaining a metabolomics profile for the first sample. In some embodiments, identifying the one or more compounds that cause the fraction shift further comprises filtering the metabolomics profile for the fraction from the second plurality of fractions to exclude compounds present in the first sample. In some embodiments, identifying the one or more compounds that cause the fraction shift further comprises (i) obtaining a metabolomics profile for the first sample; and (ii) filtering the metabolomics profile for the fraction from the second plurality of fractions to exclude compounds present in the first sample. In some embodiments, the metabolomics profiles are obtained using mass spectrometry. In some embodiments, the metabolomics profiles are obtained using liquid chromatography and tandem mass spectrometry (LC-MS/MS). In some embodiments, identifying the one or more compounds that cause the fraction shift comprises confirming co-elution of the one or more compounds and the target protein or the one or more binding proteins based on a peak elution fraction of the one or more compounds and a peak elution fraction of the target protein or the binding protein.

In some embodiments, the identifying the one or more compounds that cause the fraction shift comprises determining fragmentation profile of a natural extract. In some embodiments, the identifying the one or more compounds that cause the fraction shift comprises determining fragmentation profile of a compound library. In some embodiments, the identifying the one or more compounds that cause the fraction shift comprises determining fragmentation profile of the cellular extract and a natural extract. In some embodiments, the identifying the one or more compounds that cause the fraction shift comprises determining fragmentation profile of the cellular extract and a compound library. In some embodiments, identifying the one or more compounds that cause the fraction shift comprises determining the fragmentation profile of a natural extract and comparing it to the fragmentation profile of the cellular extract and a natural extract. In some embodiments, identifying the one or more compounds that cause the fraction shift comprises determining the fragmentation profile of a compound library and comparing it to the fragmentation profile of the cellular extract and a compound library.

### In Silico NMR/Computational NMR

In silico (computational) nuclear magnetic resonance (NMR) may be utilized to analyze a known chemical structure of a compound or a drug to calculate the likelihood of a compound binding to a protein and/or a peptide. The method predicts stable compound-protein complexes in liquid solutions by computing the nuclear magnetic resonance chemical shifts.

In some embodiments, identifying the one or more compounds that cause the fraction shift comprises obtaining a metabolomics profile for the fractions. In some embodiments, identifying the one or more compounds that cause the fraction shift comprises obtaining a metabolomics profile from the second plurality of fractions. In some embodiments, identifying the one or more compounds that cause the fraction shift comprises obtaining a metabolomics profile for the compound library or the natural extract. In some embodiments, identifying the one or more compounds that cause the fraction shift comprises identifying one or more compounds present in both the fraction and the compound library or the natural extract. In some embodiments, identifying the one or more compounds that cause the fraction shift comprises: (i) obtaining a metabolomics profile for the fraction from the second plurality of fractions; (ii) obtaining a metabolomics profile for the compound library or the natural extract; and (iii) identifying one or more compounds present in both the fraction and the compound library or the natural extract. In some embodiments, identifying the one or more compounds that cause the fraction shift further comprises obtaining a metabolomics profile for the first sample. In some embodiments, identifying the one or more compounds that cause the fraction shift further comprises filtering the metabolomics profile for the fraction from the second plurality of fractions to exclude compounds present in the first sample. In some embodiments, identifying the one or more compounds that cause the fraction shift further comprises (i) obtaining a metabolomics profile for the first sample; and (ii) filtering the metabolomics profile for the fraction from the second plurality of fractions to exclude compounds present in the first sample. In some embodiments, the metabolomics profiles are obtained using in silico NMR. In some embodiments, identifying the one or more compounds that cause the fraction shift comprises confirming co-elution of the one or more compounds and the target protein or the one or more binding proteins based on a peak elution fraction of the one or more compounds and a peak elution fraction of the target protein or the binding protein. In some embodiments, confirming co-elution comprises using in silico NMR.

### Systems

Also described herein are systems for performing the methods described herein. The system can include one or more processors and a non-transitory computer readable storage medium storing one or more programs that, when executed by the one or more processors, cause the system to perform the method. In some implementations, the system may be configured for identifying components of a protein-protein complex. In some implementations, the system may be configured for identifying one or more compounds that cause protein complex formation, stabilization, or dissociation. The system may further comprise one or more analytical components for obtaining data used in the performed method, for example, a chromatography system configured to fractionate one or more samples (which may include, for example, a size-exclusion chromatography column), one or more mass spectrometers (which may be configured to obtain proteomics data and/or metabolomics data). A system that includes one or more mass spectrometers may further include a liquid chromatography system (which may include, for example, a reverse-phase liquid chromatography column). For example, the system may include tandem mass spectrometers, which may be further equipped with a liquid chromatography system, for example to perform LC-MS/MS.

An exemplary system configured for identifying components of a protein-protein complex can include one or more processors; and a non-transitory computer readable storage medium storing one or more programs that, when executed by the one or more processors, cause the system to: receive a first proteomics profile data for a first plurality of fractions obtained by fractioning a first sample comprising a portion of a biological sample comprising proteins (for example, using size-exclusion chromatography); receive second proteomics profile data for a second plurality of fractions obtained by fractioning (for example, using the size exclusion chromatography) a second sample comprising (i) second portion of the biological sample and (ii) a compound library, a drug, or a natural extract; identify, based on the first proteomics profile data and the second proteomics data, proteins in the first plurality of fractions and the second plurality of fractions; identify, for a target protein, a fractionation shift between the first plurality of fractions and the second plurality of fractions, wherein the fraction shift indicates complex formation or stabilization, or complex dissociation, caused by the drug or one or more compounds in the compound library or the natural extract; and identify one or more binding proteins that form a complex with the target protein. Co-elution of the one or more binding proteins with the target protein in the second plurality of fractions but not the first plurality of fractions indicates that the drug or a compound in the compound library or the natural extract causes the formation or stabilization of a complex comprising the target protein and at least one of the one or more binding proteins. Co-elution of the one or more binding proteins with the target protein in the first plurality of fractions but not the second plurality of fractions indicates that the drug or a compound in the compound library or the natural extract causes the dissociation of a complex comprising the target protein and at least one of the one or more binding proteins. The co-elution of the one or more binding proteins and the target protein may be determined based on, for example, a peak elution fraction for the target protein and the one or more binding proteins. For example, co-elution of the one or more binding proteins with the target protein may be based on a peak elution fraction of the one or more binding proteins and a peak elution fraction of the target protein.

The one or more programs, when executed by the one or more processors, may further cause the system to select one or more of the one or more binding proteins as a member of the complex based on molecular weights of the one or more putative binding proteins and the target protein and a fraction number for a fraction comprising the target protein and the one or more putative binding proteins.

The system may further include an analytical system for obtaining the first proteomics profile data and/or the second proteomics data profile. For example, the system may include one or more mass spectrometers. In some implementations, the system may include a liquid chromatography system and tandem mass spectrometers, which may be configured to generate proteomics profile data using LC-MS/MS.

An exemplary system configured for identifying one or more compounds that cause protein complex formation, stabilization, or dissociation can include one or more processors; and a non-transitory computer readable storage medium storing one or more programs that, when executed by the one or more processors, cause the system to: receive a first proteomics profile data for a first plurality of fractions obtained by fractioning (for example, using size-exclusion chromatography) a first sample comprising a portion of a biological sample comprising proteins; receive second proteomics profile data for a second plurality of fractions obtained by fractioning, (for example, using the size exclusion chromatography) a second sample comprising (i) second portion of the biological sample and (ii) a compound library or a natural extract; identify, based on the first proteomics profile data and the second proteomics data, proteins in the first plurality of fractions and the second plurality of fractions; identify, for a target protein, a fractionation shift between the first plurality of fractions and the second plurality of fractions, wherein the fraction shift indicates complex formation or stabilization, or complex dissociation, caused by one or more compounds in the compound library or the natural extract; receive metabolomics data for the compound library or the natural extract; receive metabolomics data for a fraction, from the second plurality of fractions, comprising the target protein or a binding protein that forms a complex with the target protein in the absence of the one or more compounds; and identify the one or more compounds that cause the fraction shift by analyzing the metabolomics data for the compound library or the natural extract and the metabolomics data for the fraction, from the second plurality of fractions, comprising the target protein or the binding protein that forms a complex with the target protein in the absence of the one or more compounds.

In some implementations, the system is configured to identify the one or more compounds that cause the fraction shift by receiving a metabolomics profile for the fraction from the second plurality of fractions; receiving a metabolomics profile for the compound library or the natural extract; and identifying one or more compounds present in both the fraction and the compound library or the natural extract. The system may further be configured to receive a metabolomics profile for the first sample; and filter the metabolomics profile for the fraction from the second plurality of fractions to exclude compounds present in the first sample.

The system may further include an analytical system for obtaining the first proteomics profile data and/or the second proteomics data profile. For example, the system may include one or more mass spectrometers. In some implementations, the system may include a liquid chromatography system and tandem mass spectrometers, which may be configured to generate proteomics profile data using LC-MS/MS.

The system may further include an analytical system for obtaining the metabolomics profile data and/or the second proteomics data profile. For example, the system may include one or more mass spectrometers. In some implementations, the system may include a liquid chromatography system and tandem mass spectrometers, which may be configured to generate proteomics profile data using LC-MS/MS. In some embodiments, the system may include a nuclear magnetic resonance (NMR) system. For example, the system may be configured to obtain the metabolomics profile data using in silico nuclear magnetic resonance (NMR).

In some implementations, identifying the one or more compounds that cause the fraction shift can include, for example, confirming co-elution of the one or more compounds and the target protein or the one or more binding proteins based on a peak elution fraction of the one or more compounds and a peak elution fraction of the target protein or the binding protein.

In some implementations, the system is configured to identify the binding protein that forms the complex with the target protein. For example, co-elution of the binding proteins with the target protein in the first plurality of fractions but not the second plurality of fractions indicates that one or more compounds in the compound library or the natural extract causes the dissociation of a complex comprising the target protein and at the binding protein.

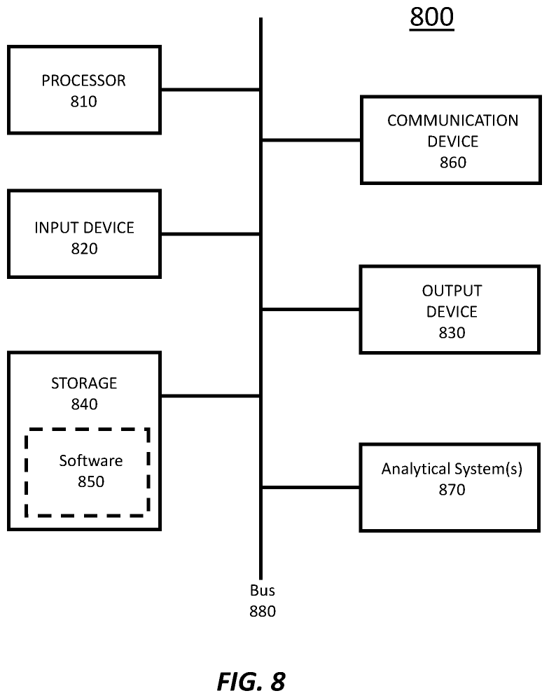

FIG. 8 illustrates an example of a computing device or system in accordance with one embodiment. Device 800 can be a host computer connected to a network. Device 800 can be a client computer or a server. As shown in FIG. 8, device 800 can be any suitable type of microprocessor-based device, such as a personal computer, workstation, server or handheld computing device (portable electronic device) such as a phone or tablet. The device can include, for example, one or more processor(s) 810, input devices 820, output devices 830, memory or storage devices 840, communication devices 860, and one or more analytical systems 870 (for example, one or more liquid chromatography systems and/or one or more mass spectrometers). Software 850 residing in memory or storage device 840 may comprise, e.g., an operating system as well as software for executing the methods described herein. Input device 820 and output device 830 can generally correspond to those described herein, and can either be connectable or integrated with the computer.

Input device 820 can be any suitable device that provides input, such as a touch screen, keyboard or keypad, mouse, or voice-recognition device. Output device 830 can be any suitable device that provides output, such as a touch screen, haptics device, or speaker.

Storage 840 can be any suitable device that provides storage (e.g., an electrical, magnetic or optical memory including a RAM (volatile and non-volatile), cache, hard drive, or removable storage disk). Communication device 860 can include any suitable device capable of transmitting and receiving signals over a network, such as a network interface chip or device. The components of the computer can be connected in any suitable manner, such as via a wired media (e.g., a physical system bus 880, Ethernet connection, or any other wire transfer technology) or wirelessly (e.g., Bluetooth®, Wi-Fi®, or any other wireless technology).

Software module 850, which can be stored as executable instructions in storage 840 and executed by processor(s) 810, can include, for example, an operating system and/or the processes that embody the functionality of the methods of the present disclosure (e.g., as embodied in the devices as described herein).

Software module 850 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 840, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 850 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

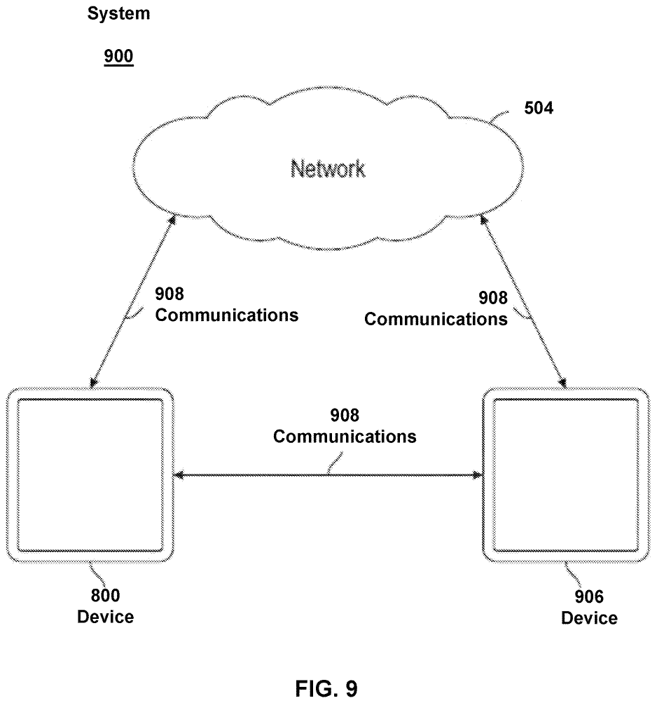

Device 800 may be connected to a network (e.g., network 904, as shown in FIG. 9 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 800 can be implemented using any operating system, e.g., an operating system suitable for operating on the network. Software module 850 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 810.

Device 800 can further include one or more analytical systems 870 (for example, one or more liquid chromatography systems and/or one or more mass spectrometers).

FIG. 9 illustrates an example of a computing system in accordance with one embodiment. In system 900, device 800 (e.g., as described above and illustrated in FIG. 8) is connected to network 904, which is also connected to device 906. In some embodiments, device 906 is an analytical system (which may include, for example, one or more liquid chromatography systems and/or one or more mass spectrometers).

Devices 800 and 906 may communicate, e.g., using suitable communication interfaces via network 904, such as a Local Area Network (LAN), Virtual Private Network (VPN), or the Internet. In some embodiments, network 904 can be, for example, the Internet, an intranet, a virtual private network, a cloud network, a wired network, or a wireless network. Devices 800 and 906 may communicate, in part or in whole, via wireless or hardwired communications, such as Ethernet, IEEE 802.11b wireless, or the like. Additionally, devices 800 and 906 may communicate, e.g., using suitable communication interfaces, via a second network, such as a mobile/cellular network. Communication between devices 800 and 906 may further include or communicate with various servers such as a mail server, mobile server, media server, telephone server, and the like. In some embodiments, Devices 800 and 906 can communicate directly (instead of, or in addition to, communicating via network 904), e.g., via wireless or hardwired communications, such as Ethernet, IEEE 802.11b wireless, or the like. In some embodiments, devices 800 and 906 communicate via communications 908, which can be a direct connection or can occur via a network (e.g., network 904).

One or all of devices 800 and 906 generally include logic (e.g., http web server logic) or are programmed to format data, accessed from local or remote databases or other sources of data and content, for providing and/or receiving information via network 904 according to various examples described herein.

### EXAMPLES

### Example 1: High-Throughput Mapping of Metabolite-Host Protein Interactions for Scalable Drug Discovery

Cell lysates were mixed with a natural extract and subject to analysis. Several proteins were identified to co-elute with RNF114 in the presence of compound. This Example demonstrates an exemplary high-throughput approach to identifying compound-protein interactions.

Lysate and extract preparation: Frozen mice tissues (pooled from brain, liver, lungs, and kidney) were homogenized in 2 mL screw-cap tubes with 1×PBS and zirconium beads in a bead-beater (speed 3450 rpm) (30 s×5 cycle). After homogenization, the lysate was centrifuged at 21,000×g for 15 minutes to obtain the clear supernatant. Protein concentration was determined using a BCA protein assay kit. Plant extracts were dissolved separately in DMSO, then pooled with mice tissue lysate (60 mg protein total).

Sample separation: The binding reaction/co-incubation incubated for 25° C. at 1 h, then subjected to size exclusion chromatography. Size exclusion chromatography was performed on an AKTA AVANT 150 (GE), Cytiva (UNICORN™ software version 7.6) using SUPERDEX 200 μg 16/600 column (GE). Running buffer for separation was 50 mM ammonium bicarbonate+150 mM NaCl in milliQ water. Prior to sample analysis, the column was equilibrated with 2 column volumes of running buffers with a flow rate: 1 mL/min. After loading the sample onto the column, the 80 collections were collected with the running buffer at flow rate: 0.8 mL/min. Quantity of protein per fraction was quantitated using a BCA assay.

Sample preparation for metabolomics: For each fraction, the volume corresponding to 20 μg total protein was sampled from the fraction (800 μL total) taken for metabolomics. Samples were dried using a speedvac, then methanol was added. Samples were sonicated for 10 mins followed by 5 mins vortex and centrifugation for 10 mins at RT. Aliquots were dried using a concentrator.

Sample preparation for proteomics: Fractions equivalent to 20 μg of protein were taken for proteomics and mixed with 20% SDS. Proteins were reduced with 5 mM tris(2-carboxyethyl) phosphine hydrochloride for 1 h at 37° C. and alkylated with methyl methanethiosulfonate (MMTS), for 30 minutes in dark at room temperature. The proteins were further digested with trypsin/lysC (1:100) overnight on a thermomixer at 37° C. Post digestion, samples were removed from the thermomixer and 0.5 μmol of PREMIS™ (Promega) peptide mix were added to samples. The peptides were then loaded onto the S-TRAP column and centrifuge at 10,000×g for 30 s. Peptides were eluted with triethylammonium bicarbonate buffer (TEABC), 0.1% formic acid and 50% acetonitrile (ACN).

Peptides were resuspended in 0.1% formic acid and proceeded for liquid chromatography with tandem mass spectrometry (LC-MS/MS) analysis. The peptides were resolved on an Ultimate 3000 RSLCnano system coupled with an Orbitrap Eclipse. 1 μg was loaded on a C18 column 50 cm, 3.0 μm Easy-spray column (Thermo Fisher Scientific). Peptides were eluted with a 0-40% gradient of buffer B (80% acetonitrile, 0.1% formic acid) at a flow rate of 300 nl/min) and injected for MS analysis. LC gradients were run for 100 minute. MS1 spectra were acquired in the Orbitrap (R=240k; AGQ target=400000; Max IT=50 ms; RF Lens=30%; mass range=400-2000; centroid data). Dynamic exclusion was employed for 10 s excluding all charge states for a given precursor. MS2 spectra were collected in either linear ion trap (rate=turbo; AGQ target=20,000; MaxIT=50 ms; NCEHCD=35%).

Data analysis: The mass spectrometry .raw files were used for proteomics database searches. Fragpipe software [Version 17.1; Releases·Nesvilab/FragPipe (github.com)] was used for proteomics database searches against the Human and Mouse Proteome database downloaded from UniProt (UniProtKB Release 2021_03), was included with contaminant and decoy proteins, for the raw data searches. The searches were conducted with precursor and fragment tolerances of 10 ppm and 0.05 Da, respectively. A false discovery rate of 0.1% was kept at both the peptide and the protein levels. The combined peptide output file was used for the dose response curve (DRC) peptide trend analysis.

The proteomics and metabolomics LC-MS/MS data were further filtered following cleanup strategies as described. Contaminants and human proteins like keratin were removed from the proteomics data, followed by removal of proteins that appear in 75% fractions and we retained proteins with at least ≥2 unique peptides and ≥2 total number of identified peptide spectra matched (PSMs) for downstream correlation analysis. Metabolomics dataset were pre-processed by removing metabolites (features) that appear in all 75% of the fractions, included metabolites (features) with abundances values >10000, and selecting metabolites/features with an associated MS/MS spectra. A false discovery rate (FDR) of 5% was calculated on the Pearson R2 values.

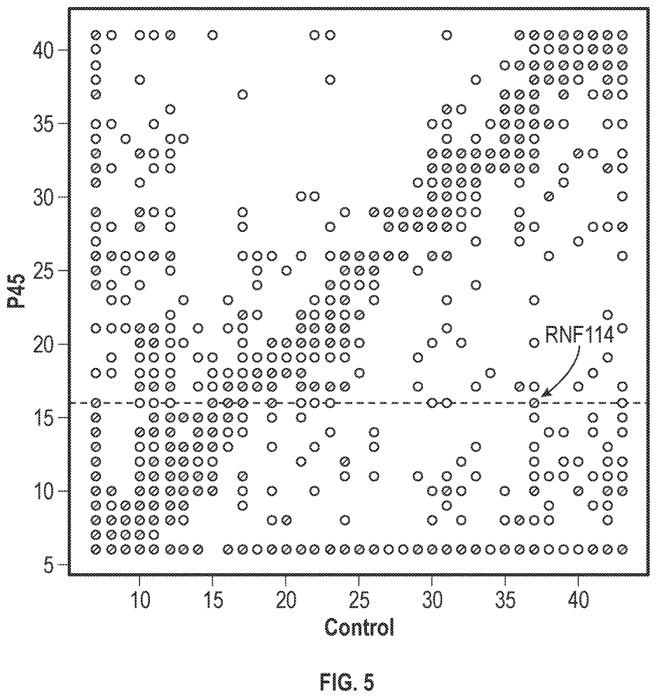

Results: Compound-protein interactions were plotted to visualize peak shifts in the presence of the compound library (FIG. 5). Proteins that did not change peak elution fractions in the presence of metabolite were visualized by on the diagonal. Proteins that moved to the right of the diagonal indicated that the protein eluted at a lower number fraction, gaining apparent molecular weight in the presence of the natural extract. Proteins that moved to the left of the diagonal indicated that the protein eluted at a higher number fraction, losing apparent molecular weight in the presence of the natural extract.

Exemplary protein RNF114 eluted in fraction 37 in mouse lysates without the metabolite library (FIG. 5). RNF114 was observed to elute in fraction 16 when lysates were incubated with the P45 plant extract (FIG. 5). This indicated that RNF114 could both form larger apparent molecular weight complexes and break into smaller apparent molecular weight complexes in the presence of P45 (FIG. 5).

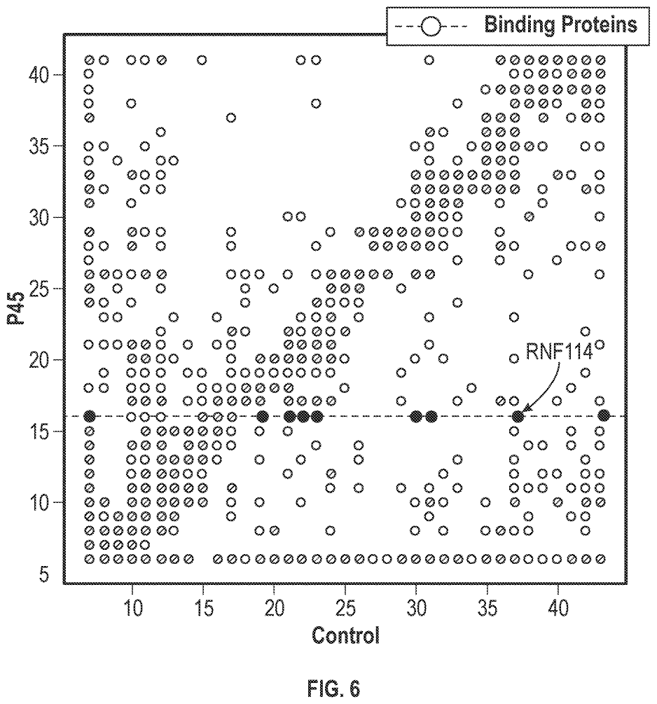

Other proteins were observed to elute in higher number fractions in the control lysate but co-elute in a lower numbered fraction with RNF114 in the presence of metabolite (FIG. 6). The shift in peak elution volume indicated that both RNF114 and binding proteins were gaining apparent molecular weight in the presence of compound. This suggested that the compound could mediate protein complex formation between RNF114 and the binding proteins.

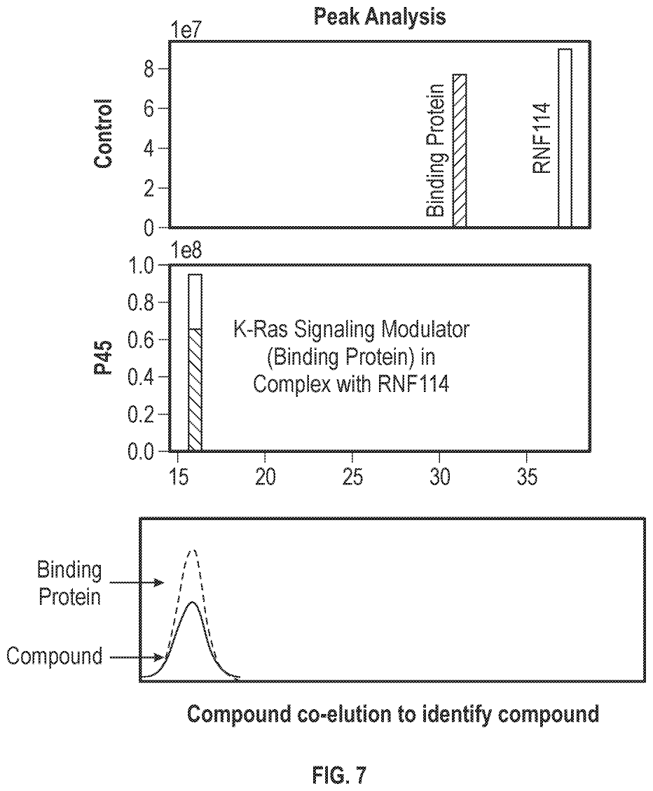

Results: To validate compound-dependent complex formation, mouse protein lysate was co-incubated with or without P45. In the absence of compound, RNF114 and a binding protein (a K-Ras signaling modulator) migrated in separate fractions (FIG. 7). In the presence of compound, the peaks corresponding to unbound proteins became undetectable in fractions 31 and 37, respectively, but appeared in fraction 16. This indicated that the P45 natural extract mediated protein-protein associations between RNF114 and the binding protein.

What is claimed is:

1. A method for identifying components of a protein-protein complex, comprising:
fractioning a first sample comprising a first portion of a biological sample comprising proteins, using size-exclusion chromatography to generate a first plurality of fractions;
fractioning a second sample comprising (i) second portion of the biological sample and (ii) a compound library, a drug, or a natural extract, using the size-exclusion chromatography to generate a second plurality of fractions;
analyzing the first plurality of fractions and the second plurality of fractions to identify proteins in the first plurality of fractions and the second plurality of fractions;
identifying, for a target protein, a fractionation shift between the first plurality of fractions and the second plurality of fractions, wherein the fraction shift indicates complex formation or stabilization, or complex dissociation, caused by one or more compounds in the compound library, the drug, or the natural extract; and
identifying one or more binding proteins that form a complex with the target protein, wherein:
co-elution of the one or more binding proteins with the target protein in the second plurality of fractions but not the first plurality of fractions indicates that a compound in the compound library, the drug, or the natural extract causes the formation or stabilization of a complex comprising the target protein and at least one of the one or more binding proteins; and
co-elution of the one or more binding proteins with the target protein in the first plurality of fractions but not the second plurality of fractions indicates that a compound in the compound library, the drug, or the natural extract causes the dissociation of a complex comprising the target protein and at least one of the one or more binding proteins.

2. The method of claim 1, further comprising selecting one or more of the one or more binding proteins as a member of the complex based on molecular weights of the one or more putative binding proteins and the target protein and a fraction number for a fraction comprising the target protein and the one or more putative binding proteins.

3. The method of claim 1 or 2, wherein co-elution is determined based on a peak elution fraction for the target protein and the one or more binding proteins.

4. The method of any one of claims 1-3, wherein co-elution of the one or more binding proteins with the target protein is based on a peak elution fraction of the one or more binding proteins and a peak elution fraction of the target protein.

5. A method for identifying one or more compounds that cause protein complex formation, stabilization, or dissociation, comprising:
fractioning a first sample comprising a first portion of a biological sample comprising proteins, using size-exclusion chromatography to generate a first plurality of fractions;
fractioning a second sample comprising (i) second portion of the biological sample and (ii) a compound library or a natural extract, using the size-exclusion chromatography to generate a second plurality of fractions;
analyzing the first plurality of fractions and the second plurality of fractions to identify proteins in the first plurality of fractions and the second plurality of fractions;
identifying, for a target protein, a fractionation shift between the first plurality of fractions and the second plurality of fractions, wherein the fraction shift indicates complex formation or stabilization, or complex dissociation, caused by one or more compounds in the compound library or the natural extract; and
identifying the one or more compounds that cause the fraction shift, comprising analyzing (1) for a fraction shift indicating complex formation or stabilization caused by the one or more compounds, a fraction from the second plurality of fractions that comprises the target protein to identify one or more compounds in the fraction that co-elutes with the target protein, or (2) for a fraction shift indicating complex dissociation caused by the one or more compounds, (i) a fraction from the second plurality of fractions that comprises the target protein to identify one or more compounds in the fraction that co-elutes with the target protein, or (ii) a fraction from the second plurality of fractions that comprises a binding protein that forms a complex with the target protein in the absence of the one or more compounds to identify the one or more compounds in the fraction that co-elutes with the binding protein.

6. The method of claim 5, wherein identifying the one or more compounds that cause the fraction shift comprises:
obtaining a metabolomics profile for the fraction from the second plurality of fractions;
obtaining a metabolomics profile for the compound library or the natural extract; and
identifying one or more compounds present in both the fraction and the compound library or the natural extract.

7. The method of claim 6, identifying the one or more compounds that cause the fraction shift further comprises:
obtaining a metabolomics profile for the first sample; and
filtering the metabolomics profile for the fraction from the second plurality of fractions to exclude compounds present in the first sample.

8. The method of claim 6 or 7, wherein the metabolomics profiles are obtained using mass spectrometry.

9. The method of claim 8, wherein the metabolomics profiles are obtained using liquid chromatography and tandem mass spectrometry (LC-MS/MS).

10. The method of claim 6 or 7, wherein the metabolomics profiles are obtained using in silico nuclear magnetic resonance (NMR).

11. The method of any one of claims 5-10, wherein identifying the one or more compounds that cause the fraction shift comprises confirming co-elution of the one or more compounds and the target protein or the one or more binding proteins based on a peak elution fraction of the one or more compounds and a peak elution fraction of the target protein or the binding protein.

12. The method of any one of claims 5-11, comprising identifying the binding protein that forms the complex with the target protein, wherein co-elution of the binding proteins with the target protein in the first plurality of fractions but not the second plurality of fractions indicates that one or more compounds in the compound library or the natural extract causes the dissociation of a complex comprising the target protein and at the binding protein.

13. The method of any one of claims 1-12, wherein analyzing the first plurality of fractions and the second plurality of fractions to identify proteins in the first plurality of fractions and the second plurality of fractions comprises a proteomics analysis.

14. The method of claim 13, wherein analyzing the first plurality of fractions and the second plurality of fractions to identify proteins in the first plurality of fractions and the second plurality of fractions comprises using mass spectrometry.

15. The method of claim 13, wherein analyzing the first plurality of fractions and the second plurality of fractions to identify proteins in the first plurality of fractions and the second plurality of fractions comprises using liquid chromatography with tandem mass spectrometry (LC-MS/MS).

16. The method of any one of claims 1-15, wherein the compound library, the drug, or the natural extract is substantially free of proteins.

17. The method of any one of claims 1-16, wherein the biological sample comprises a cell-free biological sample, a tissue extract, a cellular extract, or a sub-cellular extract.

18. The method of any one of claims 1-17, wherein the second sample comprises the compound library.

19. The method of any one of claims 1-17, wherein the second sample comprises the natural extract.

20. The method of claim 19, wherein the natural extract is a plant extract.

21. The method of any one of claims 1-20, wherein the biological sample comprising proteins is obtained from a cellular lysate.

22. The method of any one of claims 1-21, wherein the biological sample comprising proteins is obtained from animal tissue.

23. The method of any one of claims 1-22, wherein the biological sample comprising proteins is obtained from mammalian tissue.

24. The method of any one of claims 1-23, wherein the biological sample comprising proteins is obtained from brain, liver, lung, or kidney tissue.

25. A system comprising:
one or more processors; and
a non-transitory computer readable storage medium storing one or more programs that, when executed by the one or more processors, cause the system to:
receive a first proteomics profile data for a first plurality of fractions obtained by fractioning, using size-exclusion chromatography, a first sample comprising a portion of a biological sample comprising proteins;
receive second proteomics profile data for a second plurality of fractions obtained by fractioning, using the size exclusion chromatography, a second sample comprising (i) second portion of the biological sample and (ii) a compound library, a drug, or a natural extract;
identify, based on the first proteomics profile data and the second proteomics data, proteins in the first plurality of fractions and the second plurality of fractions;
identify, for a target protein, a fractionation shift between the first plurality of fractions and the second plurality of fractions, wherein the fraction shift indicates complex formation or stabilization, or complex dissociation, caused by one or more compounds in the compound library, the drug, or the natural extract; and
identify one or more binding proteins that form a complex with the target protein, wherein:
co-elution of the one or more binding proteins with the target protein in the second plurality of fractions but not the first plurality of fractions indicates that a compound in the compound library, the drug, or the natural extract causes the formation or stabilization of a complex comprising the target protein and at least one of the one or more binding proteins; and
co-elution of the one or more binding proteins with the target protein in the first plurality of fractions but not the second plurality of fractions indicates that a compound in the compound library, the drug, or the natural extract causes the dissociation of a complex comprising the target protein and at least one of the one or more binding proteins.

26. A system comprising:
one or more processors; and
a non-transitory computer readable storage medium storing one or more programs that, when executed by the one or more processors, cause the system to:
receive a first proteomics profile data for a first plurality of fractions obtained by fractioning, using size-exclusion chromatography, a first sample comprising a portion of a biological sample comprising proteins;
receive second proteomics profile data for a second plurality of fractions obtained by fractioning, using the size exclusion chromatography, a second sample comprising (i) second portion of the biological sample and (ii) a compound library or a natural extract; and
identify, based on the first proteomics profile data and the second proteomics data, proteins in the first plurality of fractions and the second plurality of fractions;
identify, for a target protein, a fractionation shift between the first plurality of fractions and the second plurality of fractions, wherein the fraction shift indicates complex formation or stabilization, or complex dissociation, caused by one or more compounds in the compound library or the natural extract;
receive metabolomics data for the compound library or the natural extract;
receive metabolomics data for a fraction, from the second plurality of fractions, comprising the target protein or a binding protein that forms a complex with the target protein in the absence of the one or more compounds; and
identify the one or more compounds that cause the fraction shift by analyzing the metabolomics data for the compound library or the natural extract and the metabolomics data for the fraction, from the second plurality of fractions, comprising the target protein or the binding protein that forms a complex with the target protein in the absence of the one or more compounds.
